(19) United States
(12) Patent Application Publication
(10) Pub. No.: US 2016/0038105 A1
(43) Pub. Date: Feb. 11, 2016

(54) IMAGING SYSTEM AND METHOD

(71) Applicant: ZUMA DENTAL, LLC, Los Angeles, CA (US)

(72) Inventors: Robert Hayman, Los Angeles, CA (US); Ekram Khan, League City, TX (US); Michael Paloian, Cold Spring Harbor, NY (US); Anthony Orchard, Wantagh, NY (US)

(21) Appl. No.: 14/776,706

(22) PCT Filed: Sep. 29, 2013
(86) PCT No.: PCT/US2013/062503

§ 371 Date: Sep. 14, 2015

Related U.S. Application Data
(63) Continuation-in-part of application No. 29/449,872, filed on Mar. 15, 2013, now Pat. No. D739224.
Application No. 29/449,872 is a continuation-in-part of application No. 29/450,036, filed on Mar. 15, 2013, now Pat. No. D703322.
Application No. 29/450,036 is a continuation-in-part of application No. 29/449,938, filed on Mar. 15, 2013, now Pat. No. D703324.
Application No. 29/449,938 is a continuation-in-part of application No. 29/449,918, filed on Mar. 15, 2013, now abandoned.
Application No. 29/449,918 is a continuation-in-part of application No. 29/449,957, filed on Mar. 15, 2013, now Pat. No. D703325.
Application No. 29/449,957 is a continuation-in-part of application No. 29/449,965, filed on Mar. 15, 2013, now Pat. No. D713039.
Application No. 29/449,965 is a continuation-in-part of application No. 29/449,978, filed on Mar. 15, 2013, now Pat. No. D703326.
Application No. 29/449,978 is a continuation-in-part of application No. 29/449,990, filed on Mar. 15, 2013, now Pat. No. D710501.
Application No. 29/449,990 is a continuation-in-part of application No. 29/450,009, filed on Mar. 15, 2013, now Pat. No. D710502.
Application No. 29/450,009 is a continuation-in-part of application No. 29/450,023, filed on Mar. 15, 2013, now Pat. No. D703327.
Application No. 29/450,023 is a continuation-in-part of application No. 29/466,712, filed on Sep. 11, 2013, now Pat. No. D703323.
Application No. 29/466,712 is a continuation-in-part of application No. 14/027,234, filed on Sep. 15, 2013.
(60) Provisional application No. 61/800,445, filed on Mar. 15, 2013.
(60) Provisional application No. 61/878,579, filed on Sep. 16, 2013.

(51) Int. Cl. A61B 6/14; A61B 6/00; A61B 6/06

(52) U.S. Cl. CPC A61B 6/145; A61B 6/06; A61B 6/4283

(57) ABSTRACT

The present invention is related to system and method for capturing dental images, for example, an aiming and positioning device for capturing dental images of a patient's teeth using a collimator. The system includes an image receptor holding device with an elongated arm adapted to receive and couple the receptor holder to a collimation structure. The elongated arm extends between the collimator and the receptor holder with one end coupling to the rear portion of the receptor holder and another end coupling to the collimator, and aligns the receptor with the collimator opening in an unobstructed manner. A biting portion or surface is present on the elongated arm adjacent to the receptor holder such that when the receptor is positioned behind the teeth on which the images are being taken, the biting surface is gripped by the teeth of the patient.

### CROSS-REFERENCE TO RELATED APPLICATIONS

This is a U.S. national stage application of a PCT international application no. PCT/US13/062503, filed Sep. 29, 2013, entitled “IMAGING SYSTEM AND METHOD”, which claims the priority and benefit of U.S. provisional patent application Ser. No. 61/800,445, filed Mar. 15, 2013, entitled “IMAGING SYSTEM AND METHOD”; and U.S. provisional patent application Ser. No. 61/878,579, filed Sep. 16, 2013, entitled “IMAGING SYSTEM AND METHOD”; the contents of all of which are hereby incorporated by reference in their entirety.

This application is a continuation-in-part of the following: U.S. design patent application Ser. No. 29/449,872, filed Mar. 15, 2013, entitled “CLAMP”, now allowed; U.S. design patent application Ser. No. 29/449,918, filed Mar. 15, 2013, entitled “ATTACHMENT DEVICE”, now allowed; U.S. design patent application Ser. No. 29/449,938, filed Mar. 15, 2013, entitled “X-RAY AIMING DEVICE”, now U.S. Design Pat. No. D703,324; U.S. design patent application Ser. No. 29/449,957, filed Mar. 15, 2013, entitled “RECEPTOR HOLDING DEVICE”, now U.S. Design Pat. No. D703,325; U.S. design patent application Ser. No. 29/449,965, filed Mar. 15, 2013, entitled “RECEPTOR HOLDING DEVICE now U.S. Design Pat. No. D713,039”; U.S. design patent application Ser. No. 29/449,978, filed Mar. 15, 2013, entitled “RECEPTOR HOLDING DEVICE”, now U.S. Design Pat. No. D703,326; U.S. design patent application Ser. No. 29/449,990, filed Mar. 15, 2013, entitled “RECEPTOR HOLDING DEVICE”, now U.S. Design Pat. No. D710,501; U.S. design patent application Ser. No. 29/450,009, filed Mar. 15, 2013, entitled “RECEPTOR HOLDING DEVICE”, now U.S. Design Pat. No. D710,502; U.S. design patent application Ser. No. 29/450,023, filed Mar. 15, 2013, entitled “RECEPTOR HOLDING DEVICE”, now U.S. Design Pat. No. D703,327; U.S. design patent application Ser. No. 29/450,036, filed Mar. 15, 2013, entitled “COLLIMATOR”, now U.S. Design Pat. No. D703,322; and U.S. design patent application Ser. No. 29/466,712, filed Sep. 10, 2013, entitled “COLLIMATOR STRUCTURE”, now U.S. Design Pat. No. D703,323; and U.S. utility patent application Ser. No. 14/027,234, filed Sep. 15, 2013, entitled “IMAGING SYSTEM AND METHOD”; the contents of all of which are hereby incorporated by reference in their entirety.

### FIELD OF THE INVENTION

The present invention is related to systems, devices and methods for capturing dental and/or medical images of patients. For example, the invention relates to an imaging system, devices and method for capturing medical and/or dental images of a patient's teeth and/or oral cavity.

### BACKGROUND

Dentists and oral surgeons generally used x-radiation (“x-rays”) to capture images of their patients' teeth, mouths and gums to aid in diagnosis of a patient. Traditionally, oral and dental images are captured on radiographic film placed in the patient's mouth, for example behind a patient's tooth, and an x-ray beam is projected through the tooth and onto the film. More recently, film-less or electronic sensor dental imaging has been used to capture images. The X-ray beam is projected through the patient's teeth in the same manner as in the film based methods, and an electronic sensor is placed in the patient's mouth behind the tooth to be examined. The electronic sensor may include a charge-coupled device (CCD), a complementary metal oxide semiconductor (CMOS), or any other film-less radiation sensor. These electronic sensors convert the x-rays into an electrical signal, which is often transmitted over a wire to a computer, either directly or though a module containing intermediate processing circuitry. The computer processes the signal to produce an image on an associated output device, such as a monitor or a printer.

Various devices exist in the art for capturing medical and dental images of a patient. Such devices typically have separate parts and pieces cooperating with each other to produce such images during operation. Having a system which parts and pieces cooperate better than now in existence during operation may be desirable to aid in better capturing such images and minimizing exposure of patients to radiation unnecessarily.

### SUMMARY OF THE INVENTION

The present invention relates to a system, method and device for capturing dental and/or medical images. The images may be captured on and/or by a receptor which may include, for example, film or an electronic sensor such as a digital sensor or a camera. The system includes separate parts and devices, which may all cooperate to produce dental and/or medical images with improved aiming and/or alignment, provide a substantially unobstructed line of sight between the radiation source, such as the X-ray emitter and the receptor, such as the X-ray film or electronic sensor including a digital sensor or camera, and requiring minimal assembly or adjustment during and/or just prior to actual operation to acquire images. The system, method and device are capable of producing better quality images, reducing retakes, and/or reducing artifacts, thus minimizing exposure of patients to radiation unnecessarily with any conventional radiation source already in use.

The system may include an emitter tube, which may be for example, any existing emitter tubes such as an X-ray tube, a positioning and aiming device, which may include a collimator or a collimator structure that may be removably attached to the radiation source, for example, an X-ray emitter tube, and to a receptor, which may be positioned in a holding device or is self supporting The components of the system cooperate to provide a substantially unobstructed line of sight between the radiation source, such as the X-ray emitter and the receptor, such as the X-ray film or electronic sensor including a digital sensor or camera which may be positioned in a holding device or is self supporting, as mentioned above. The unobstructed line may also, for example, be substantially orthogonal to the receiving surface of the receptor and/or substantially parallel to the emitting axis of a radiation source with minimal obstruction or interference from the structure of the receptor holder for holding the film or electronic sensor. The holding device, if present, has an elongated arm that may include curves for better achieving the unobstructed line of sight. The holding device may include some adjustability, for example, having separate parts connecting together, or using different integral holding devices, one suitable for imaging different parts of, for example, a patient's oral cavity, such as, for imaging the front, the anterior or the posterior, etc., to achieve the unobstructed line of sight. Adjustability through connection may include modular components of the holding device that are easily snapped together with complementary connection features to form holding devices suitable for different views of the oral cavity, using same modular components or different modular components, if needed.

In one exemplary embodiment of the invention, the imaging system may include a positioning and aiming device with improved connection features between the collimator or collimator structure and the emitter tube, such as an X-ray tube. The collimator structure may include a collimator having a body or frame, for example, a tube-shaped device having a length, or a plate-like device having a thickness, and an opening and/or aperture therein. The collimator or collimator structure may be attached to the radiation emitter source through a secure (during use) and yet easily removable (when not in use) attachment system that may be internal or external to the emitter tube. The attachment system may be adjustable to fit various circumferential spans of the emitter tubes, or may include different attachment systems each specific to one of different circumferential spans of the emitter tubes. The system is adapted for aligning a radiation source, such as an X-ray emitter with a receptor, through the collimator opening and/or aperture, to capture dental images of a target such as a patient's teeth, on and/or with the receptor, in an unobstructed line of sight which may include, for example, film or an electronic sensor such as a digital sensor or a camera, as noted above. The receptor may also be positioned in a receptor holding device, which may include, for example, a curved arm portion. In one example, the imagining system adapted for imaging an oral cavity includes a collimator structure having a central frame with an attachment mechanism on one end of the central frame. The attachment mechanism may include, for example, biasing arm-like structures adapted for attaching the collimator structure to an emitter tube, with the frame having an outside surface and an aperture therein. An aperture adjustment mechanism may be disposed in the frame for varying a size and/or shape of the aperture of the collimator structure and a receptor holding device for holding a receptor, the holding device may include an elongated arm having a curved portion with an attachment portion disposed on the elongated arm towards one end of the arm and a receptor holder portion disposed on the elongated arm towards a second end of the arm. The outside surface of the frame may include a plurality of features with at least one of the plurality of features being adapted for magnetically mating with the attachment portion on the arm of the receptor holding device to create an unobstructed line of sight between the aperture and the receptor during imaging.

The plurality of features of the frame are complementary to the features on the attachment portion on the arm of the receptor holding devices, with at least one of which adapted for magnetically mating the collimator structure and the attachment portion on the arm of the receptor holding device. In addition, the plurality of features may be adapted for separately mating with different receptor holding devices to accommodate different types of receptor holding devices. This may be accomplished with a sliding feature or attachment features disposed at different locations on the frame.

In another exemplary embodiment of the invention, the positioning and aiming device may include improved connection features between the collimator or collimator structure and the emitter tube, such as an X-ray tube. The collimator structure may include a collimator having a body or a frame, for example, a tube-shaped device having a length, or a plate-like device having a thickness, and an opening and/or aperture therein. The collimator or collimator structure may be attached to the radiation emitter through a secure (during use) and yet easily removable (when not in use) attachment system that may be internal or external to the emitter tube. The attachment system may be adjustable to fit various circumferential spans of the emitter tubes, or may include different attachment systems each specific to one of different circumferential spans of the emitter tubes. The system may include an aiming ring positioned between the collimator and the receptor, adapted for aligning the radiation source, such as an X-ray emitter with a receptor, through the collimator opening and/or aperture, in an unobstructed line of sight, to capture dental images of a target such as a patient's teeth, on and/or with the receptor, which may include, for example, film or an electronic sensor such as a digital sensor or a camera, as noted above. The receptor may also be positioned in a receptor holding device which may include, for example, a curved arm portion. In one aspect, the aiming ring may be a separate structure attached to the collimator structure and the receptor. In another aspect, the aiming ring may be an integral part of the collimator structure. In one example, the imagining system adapted for imaging an oral cavity includes a collimator structure having a central frame with an attachment mechanism on one end of the central frame. The attachment mechanism may include, for example, biasing arm-like structures adapted for attaching the collimator structure to an emitter tube, the frame having an outside surface and an aperture therein. An aperture adjustment mechanism may be disposed in the frame for varying a size and/or shape of the aperture of the collimator structure, and a receptor holding device for holding a receptor, the holding device may include an elongated arm having a curved portion with an attachment portion disposed on the elongated arm towards one end of the arm and a receptor holder portion disposed on the elongated arm towards a second end of the arm. An aiming ring portion may be positioned at one end of the frame for attachment to the receptor holding device. The aiming ring may include a plurality of features, at least one of the features being adapted for magnetically mating with the attachment portion on the arm of the receptor holding device. In one aspect, the outside surface of the frame may also include a plurality of features with at least one of the features being adapted for magnetically mating with the plurality of features on the aiming ring. In another aspect, the aiming ring may be an integral part of the frame. The imaging system together creates an unobstructed line of sight between the aperture and the receptor during imaging.

At least one of the plurality of features of a separate aiming ring may include features complementary to the features on the frame and the attachment portion on the arm of the receptor holding devices for magnetically mating with the housing and the attachment portion on the arm of the receptor holding device. In addition, the plurality of features on the aiming ring may be adapted for separately mating with different receptor holding devices to accommodate different types of receptor holding devices. This may be accomplished with a sliding feature or attachment features disposed at different locations on the ring. Also, at least one of the plurality of features on the aiming ring may include a handle for facilitating easy handling and connection.

In one exemplary embodiment, a collimator or collimator structure may in general be a reversibly removable attachment to an existing separate radiation source, such as an x-ray source. In one embodiment, the collimator may be removably attached to the source, for example, the X-ray emitter tube, with an attachment mechanism that surrounds the outside of, for example, the X-ray emitter tube. In another embodiment, the collimator may be removably attached to the source, for example, the X-ray emitter tube, with an attachment mechanism internal to the, for example, X-ray emitter tube. In still other embodiments, the collimator may be attached to and extends from the end of an X-ray emitter tube, with an attachment system, for example, that may includes an attachment system that is internal or external to the emitter tube.

An internal attachment or external attachment mechanism for attaching the collimator or collimator structure may be adapted for accommodating different sizes of the emitter tube of the radiation source, for example, the X-ray emitter tube. The mechanism for accommodation may include one that is an integral feature of the attachment system or an additional feature that may aid in the accommodations.

In one embodiment of the invention, the internal attachment mechanism includes a substantially cylindrical shaped tube-like structure with a central cylindrical frame having three identical substantially rectangular window-like apertures or depressions arranged equal distance from each other. Three arms, for example, three arc shaped sections when the radiation source, for example, the X-ray emitter tube, is cylindrical in shape, are attached to the central frame of the, for example, substantially cylindrical tube-like structure, to cooperate in the closed position, i.e., when not in use, to substantially close the window-like aperture to form the substantially cylindrical shaped tube-like structure. The arms, if they are of arc-shaped portions, may include an arc shaped outer surface. When mounted, the arms may pivot or swing on fixed pivot points in the cylindrical frame and expand radially from the central cylindrical frame. The arms may be mounted for pivoting or swinging from a closed position to a partly open position, for example, for substantially the same angle or distance from the central frame for all arms. The angles of outward swing of the arc shaped portions determine the outer diameter of the attachment mechanism and may be adjusted to fit the internal diameter of an X-ray emitter tube, for a cylindrical shaped tube. The arc shaped portions or arms may be biased so that in the open position, the outside surfaces of the arms rest and press against the inside surface of the radiation source, for example, the X-ray emitter tube, to achieve frictional retention. The biasing may be effected by springs or any other types of biasing structure. For example, leaf springs, coil springs, and/or any other appropriate springs may be utilized. Coil springs may generally be utilized with swinging arms by the torsioning of the spring. The springs, if used, may be any metal or polymeric spring having sufficient spring force to affect a secure attachment when the arms are swung to any open position. Though the arms are disclosed as arc shaped portions having an arc shaped outer surface, the shape of the outer surface maybe any other shape and is dictated substantially by the shape of the inner surface of the radiation source, for example, the X-ray emitter tube to ensure a secure attachment, and/or by the shape of the attachment mechanism of the collimator as a whole.

In one embodiment, each of the substantially arc shaped arms or sections may include tabs or similar structures extending vertically from each end, along one edge of each of the arms. The attachment of the arms maybe effected by mating these tabs or similar structures to cavities or channels on the upper and lower horizontal edges of the window-shaped apertures or openings so that the arms or portions are mounted vertically in the central frame structure. The mating allows each of the substantially arc shaped portions to pivot or swing.

In another embodiment, each of the substantially arc shaped arms or portions may include an internal lumen extending vertically along one edge of each of the arms. A rod having the correct shape and dimension may be mounted inside the lumen and extending past the ends of the arms. The rod may be securely and fixedly mounted in place in the cavities or channels present on the upper and lower horizontal edges of the window shaped apertures or openings so that the arms or portions are mounted vertically. The pivot or swing motion may be facilitated by the mating of the rod inside the internal lumen.

In a further embodiment, the arms may be mounted on the central frame similar to mounting a door, such as with multiple alternating lumens on the arm and frame through which a rod may be disposed for pivoting or swinging.

The non-pivotal ends of the arms may be disposed for rotation to draw the non-pivotal ends of the arms radially inward or outward from the central cylindrical frame. For example, the arms may include, for example, pins which may rest within radially arcuate slots in a plate, which may be rotationally coupled against a second plate, both of which may include handles for rotating the plates in opposite angular directions to causes the pins of the arms to translate within the slots, which in turn draws the non-pivotal ends of the arms 104 radially inward or outward from the central cylindrical frame.

To effectuate the internal attachment mechanism, the arms may be held in a contracted position to insert them into the X-ray tube. Once inside the tube, the arms are allowed to expand, causing the outer surfaces of the arms to rest against the inside surfaces of the X-ray tube. The attachment is secured by the biasing springs and held securely in place by friction.

The arms may also generally include friction-enhancing features, such as being constructed from a high frictional material, having a coating of high frictional material, and/or having an attachment of a high frictional material such that the surface that touches the inside of the radiation source may have enhanced frictional engagement.

In an alternative embodiment, arms may be utilized that extend out radially orthogonally from a central frame. Biasing elements, such as springs discussed above, may be used and may be generally oriented orthogonally to the surface of the central frame such that they push the arms directly outward in opposition to compression of the spring, rather than in opposition to torsion in of the spring as above.

In another embodiment of the invention, an internal connection mechanism may include a central frame having spirally arranged portions nested together prior to use and expanded to the proper connection size during use. The spirally arranged portions are arranged such that the portions may be rotated in one direction to expand the distance between the portions to fit into the large circumferential emitter tubes and in the other direction to contract the distance between portions to fit into the smaller circumferential size emitter tubes. The spirally arranged portions may include features to lock the device in place once a proper size is found.

In a further embodiment of the invention, an external attachment mechanism may include a substantially cylindrical shaped tube having two halves hingedly connected along one edge of each panel parallel to the long axis of the tube. The two halves cooperate to surround the emitter tube and the collimator when one end of the emitter tube abuts one end of the collimator, with a fixing or locking mechanism or features, such as complementary locking features on the free edges of each half. The external attachment mechanism may be adjustable to accommodate different sizes of the radiation source such as, for example, the X-ray emitter tube when each half is made of material that may be expandable and contractible, similar to a spring loaded accordion material that extends and contracts to accommodate varying sizes of the emitter tube and matching collimator. In general however, different attachment devices and collimators may be used for emitter tubes with different diameters and/or shapes. The material for the attachment mechanism may be radiation attenuating, radiation absorbing or substantially radiation blocking to prevent radiation from escaping in the connection region.

In yet another embodiment of the invention, the external attachment mechanism may include a cylindrical tube that may be radially adjusted to expand and contract to accommodate different sizes of the radiation source, for example, the X-ray emitter tube. The mechanism may include overlapping blades radially arranged to expand and contract similar to an iris. In one aspect, the tube may be permanently attached to one end of the collimator. In another aspect, the tube may be separate from the collimator.

In still another embodiment of the invention, the attachment device may include short petal-like portions mounted on one end to the central structure which may be the collimator, at the end of the collimator to be attached to the emitter tube and biased for expansion of contraction in the free end. The petal-like portions may be spaced apart and overlap each other during use.

In still yet another embodiment of the invention, the external attachment mechanism may include discrete claw-like and/or jaw-like portions mounted on one end to the central structure which may be the collimator, at the end of the collimator to be attached to the emitter tube and pivoted for movement, such as for expansion of contraction, in the free end. In one aspect, there may be at least two claw-like portions. In another aspect, there may be at least three claw-like portions. In a further aspect, there may be four claw-like portions. These portions claw onto the outside surface of the emitter tube during use.

The claw-like or petal-like portions attached to the outside surface of the emitter tube. For better attachment, the inside surface of the petal-like or claw-like portions may be arc-shaped to conform better to the outside surface of the emitter tube.

In yet a further embodiment of the invention, the external attachment mechanism may include a spirally wound device that may expand or contract circumferentially to fit over the size of the emitter tube used and grasp the emitter tube by pressing inward. The spiral may be constructed of a length of ribbon-like material, located on a central frame which may have tracks. By twisting and/or torsioning the ribbon-like material, the spiral expands or contracts in circumferential and/or radial sizes to accommodate the different circumferential size of emitter tubes. This mechanism may also be utilized as an internal attachment mechanism having similar spirally wound components, where the spirally wound device may expand or contract circumferentially to fit the inner size of the emitter tube used and push outward against the inside of the emitter tube.

In still yet a further embodiment of the invention, the attachment mechanism may include a central frame having a plurality of concentric cylindrical tubes telescopically connected and biased. The telescopically arranged portions may extend and contract lengthwise to accommodate various sizes of the emitter tube and may translate within each other in a manner similar to an expanding telescopic antenna, and thus the portions may be drawn out until an appropriately sized portion is exposed. Once the proper size portion is exposed, the device is fixed with a fixing feature, such as a latch, clip or screw, to keep the telescopic portions from collapsing. The outside of the telescopic portion may be used to frictionally engage the inside of an emitter tube or the inside of the telescopic portion may be used to frictionally engage the outside of the emitter tube.

In general, a collimator may alter the radiation output from a radiation source such that the radiation is substantially collimated or traveling largely or substantially in a single direction which may be parallel to a central axis of the collimator and/or the unobstructed line as above. The size and spread of the beam of radiation emanating from the radiation source, for example, the x-ray emitter tube, may be adjusted to any shape, for example, a substantially rectangular shape of varying sizes by a collimator structure including the collimator. This may be desirable to align the radiation to be incident on the receptor while minimizing scatter and excess radiation not used to expose the receptor.

In another exemplary embodiment, the present invention also relates to an aperture adjustment mechanism for varying the size and/or shape of the aperture of the collimator or collimator structure. The mechanism includes a simple adjustment mechanism employing a few parts for varying the size and/or shape of the opening of the collimator or collimator structure and may include radiation absorbing, attenuating and/or blocking properties.

In general, the collimator itself may include an outer shell for defining the outline of the collimator itself, and the beam may be varied using a simple adjustment mechanism employing a few parts for varying the size of an opening of the collimator. In general, a radiation attenuating, absorbing and/or otherwise blocking material may be utilized to stop at least a portion of the radiation from passing through an aperture or opening in the collimator. A primary blocking component may generally prevent radiation from passing through the collimator from a radiation source except through an aperture and may generally define the maximum size and shape for a radiation beam passing through the aperture. An adjustment mechanism utilizing other and/or additional blocking components may also be utilized to vary the shape and size of the radiation beam that passes through the aperture. In general, at least two blocking components with openings and/or apertures may be utilized by varying the overlap of the openings and/or apertures.

For example, the aperture adjustment mechanism of the collimator may include, for example, three x-ray absorbing or attenuating plates of different sizes, cooperating together to vary the size and/or shape of the opening or aperture. The parts defining the opening or aperture may include an aperture control, for example, a rocker, a handle, a dial or a lever, and three plates, for example, radiation attenuating plates, which cooperate to vary the size and/or shape of the opening or aperture for the radiation from the source to pass through. The three plates may include a larger fixed plate, and two smaller movable plates, for example, stacked on top of the fixed plate. The size of the collimator opening or aperture may be adjusted using the aperture adjustment mechanism, to any desired dimension and may generally be a function of the size and shape of the apertures in the plates. The larger fixed plate may block substantially all radiation if desired and thus radiation only passes though the aperture with minimal stray or unwanted radiation coming through the collimator. This may be especially desirable at the edges of the collimator near the interface with the x-ray or other radiation source. The two inner plates with overlapping apertures may be positioned at discrete positions for a user to choose aperture size and/or shape. For example, three discrete positions may be used by moving an aperture control, such as a rocker, a handle, a dial or a lever to generate the desired aperture. The aperture control may, for example, move both plates simultaneously in opposite directions through an arc described by three points. The three points of the arc define three different size apertures. Two arced slots may be located on one side of each plate which may aid in providing parallelism and confined alignment. Stationary pins may then be nested in the arced slots such that the plates may move in the desired path of the arcs.

The outer shell defining the external outline of the collimator may include one shell or two shells. In one aspect, the shell or shells may have a small length, for example, each in the form of a thick plate. In another aspect, the collimator may have some length defining a short tubular portion.

In a further exemplary embodiment, the present invention includes a self-guiding attachment and aligning feature including the use of, for example, a magnetic material or element, for aligning the collimator or collimator structure with the receptor holding device to facilitate the imaging process. The collimator or collimator structure may also include multiple attachment or aligning features for attaching the receptor holding device at different positions to achieve different orientations of the receptor holding device relative to the collimator or collimator structure. For example, two attachment or aligning features may be present on the collimator or collimator structure at substantially 90 degrees separation such that the receptor holding device may be attached at a horizontal or vertical orientation relative to the collimator or collimator structure. It may be appreciated that these arrangements may produce a variety of different orientations of the receptor depending on which attachment or alignment feature and which receptor holding device is used.

In one embodiment, the magnetic material or element may be partially or wholly embedded or disposed on the outside, in predetermined position, of the components to be mated. This may facilitate and promote repeatable positioning in predetermined locations or spots. The location or spot may include complementary features, for example, indents and protrusions fitting into the indents for secure connection.

In another embodiment, the magnetic material or element may be partially or wholly embedded or disposed on the outside, in predetermined position of the components to be mated, each component having a feature that may be the same or complementary, and a separate part having magnetic properties with complementary features to one or the other components to be mated to facilitate coupling of the components.

The magnetic material or element may be of sufficient strength to hold the components in place once mated, but not strong as to make the separation of the components too difficult. For example, the attachment may be firm enough to resist movement by the patient or user, but a twist or a firm pull may be sufficient to effect separation. The magnetic material or element may include, for example, permanent magnets, electromagnets, rare earth element magnets, magnetically responsive materials, such as ferromagnetic metals or compounds, magnetizable materials, and/or any other appropriate magnetic material or combinations thereof. The magnet, magnetic element or magnetic material may also be mixed with polymers or dispersed in a polymer matrix. For example, some components may include a magnet or magnetic material and the mating complementary component may include a metallic part embedded or disposed on the outside of the component or piece which may be attracted to the magnet. This may be desirable as the polarities of multiple magnets may cause unhelpful interference with each other. Also, a magnet or magnetic material on one complementary component may attract a metallic part or magnetic sensitive part embedded or disposed on the outside of the component or piece on another component while still allowing a greater degree of movement, whereas multiple magnets may generally force themselves to align with each other in a particular position or alignment of maximized magnetic forces, which may make sliding components for different positions more difficult.

In any embodiment, the spot or location may vary in size or dimension. A larger dimension may provide more secure connection, provided it is not obtrusive or aesthetically unpleasing.

In a further exemplary embodiment, the present invention further relates to a system having above mentioned improvements to facilitate operation. The positioning and aiming device may include a collimator that may be removably attached to the radiation source, for example, an X-ray emitter tube, and to a receptor. The components of the system cooperate to provide a substantially unobstructed line of sight between a radiation source, such as the X-ray emitter and the receptor, for example, the X-ray film or electronic sensor including a digital sensor or camera. The unobstructed line may also, for example, be substantially orthogonal to the receiving surface of the receptor and/or substantially parallel to the emitting axis of a radiation source with minimal obstruction or interference from the structure of the receptor holder for holding the film or electronic sensor. The holding device may include an elongated arm that includes curves for better achieving the unobstructed line of sight. A separate version holding device may be used for different views of, for example, the oral cavity, to achieve the unobstructed line of sight, though devices with adjustability may also achieve the goal. In some embodiments, an aiming ring may be used alone without the collimator for imaging some spots of the oral cavity that may not be amenable to having a collimator.

In yet a further exemplary embodiment, the present invention relates to integrated receptor holding devices that minimize assembly right before use to improve efficiency and faster and better alignment. The integrated holding device may include different versions for different views of the, for example, a patient's oral cavity, such as anterior, posterior, panoramic, etc. The receptor may be removably and/or reversibly mounted in a receptor holder, such as with a holder that is reusable; or the receptor and receptor holder may be removably mounted on the positioning and aiming device if the receptor holder is not reusable. The receptor holder holds the x-ray film or digital sensor or camera and for dental x-ray use in an unobstructed manner, for example, without intruding into the active portion of the receptor, while portions of the receptor holder and film or digital sensor or camera may be inserted into a patient's oral cavity during imaging.

In still a further exemplary embodiment, the present invention relates to modular receptor holding devices that includes some parts, for example, the curved arm portion, that may be used to create different versions of the receptor holding devices that may be used for different views of the, for example, a patient's oral cavity. The modular parts may include complementary connecting features for ease of connection, for example, to be snapped together, Thus, the modular parts that may be used for multiple versions of the receptor holding device may minimize the number of different devices to be manufactured. For example, a curved arm portion may be connected to a portrait sensor holder as well as a landscape sensor holder.

In yet still a further exemplary embodiment, the present invention, in addition to modular components of the sensor holding devices, as mentioned above, that may be connected to form the various versions of the sensor holding device, may also include holding devices that may have some adjustability, for example, of the curve portion. Minimal flexing may also be desirable in holding arms that may be adjustable. Adjustability may be effected by, for example, a rotating mechanism or a connecting mechanism, articulating together to generate different versions. For example, a rotation of the arm portion may create a different version of the receptor holding device.

In one aspect of the invention, the receptor, such as the film or sensor may be removably mounted on a receptor holding device, a portion of which, if used in dental X-rays, may be inserted into a patient's oral cavity. The receptor holding device may include an elongated arm with the receptor holder on one end and a collimator connection portion on the opposite end. Coupling with the collimator may generally be desirable to achieve proper alignment such that the radiation passes through the collimator and incidents on the receptor without excess scatter and/or without passing through any unwanted structures. Examples of unwanted structures may include portions of the receptor holder that may intrude into the pathway.

According to one exemplary embodiment, the receptor holder and the elongated arm may be separate and cooperate to receive and couple the receptor holder to the collimator. The elongated arm extends between the collimator and the receptor holder with one end coupling to the rear portion of the receptor holder and another end coupling to the collimator at the connection portion to align the receptor with the collimator opening in an unobstructed line of sight arrangement. As noted above, the elongated arm may also include a curved portion in the form of, for example, an s-shaped curve. This curve portion may be adjustable or different for different receptor holding devices for imagining different parts of an oral cavity. A biting surface may be present on the elongated arm adjacent to the receptor holder such that when the receptor is positioned behind the teeth on which the images are being taken, the biting surface may be gripped by the teeth on the opposite side of the patient's oral cavity. The elongated arm and receptor holder include features articulating together for consistently centering the receptor holder with the collimator in an unobstructed line of sight arrangement so that substantially all of the radiation emanating from the collimator is captured by the receptor positioned in the receptor holder regardless of the size of the receptor used.

As noted above, in some situations, an aiming ring may be used in place of the collimator to align the receptor with the radiation source in an unobstructed line of sight arrangement.

According to one embodiment, the coupling features may be magnetic which cooperate to connect the receptor holder to the elongated arm and aid in centering the receptor holder with the collimator. The magnetic features may be partially or fully embedded in or disposed on the outside of the components to be connected or mated, as mentioned above for other magnetic features.

According to another embodiment, the features may include slots and stops so that when fitting the stops in the slots, it aids in centering the receptor holder with the collimator. The slots or stops may also include magnetic properties to further facilitate the connection.

According to another exemplary embodiment, the receptor holding device may include a bite portion adjacent to the receptor holder for removably retaining a receptor, for example, a film or a digital sensor, and a connection portion for connecting the arm to a collimator disposed along the length of the elongated arm at a distance from the bite portion. The bite portion, as its name, may be bit by the patient during imaging.

According to a further exemplary embodiments, the elongated arm of the receptor holding device may integrally include a bite portion, a receptor holder for removably retaining a receptor, such as a film or a digital sensor, adjacent to the bite portion, an aiming ring portion disposed along the length of the arm at a distance from the bite portion, and a connection portion for connecting to a collimator disposed along its length adjacent to the aiming ring portion. According to one aspect, the receptor holding device may be of a one-piece design, integrally formed with the receptor holder, bite portion and collimator attachment portion. According to another aspect, the receptor holder may be separate and not integrally formed with the elongated arm and may be attached to the arm during use. In one aspect, the aiming ring may be a separate component connected to the receptor holding arm at the aiming ring portion. In another aspect, the aiming ring may be integrally formed on the aiming ring portion of the elongated arm. In a further aspect, the aiming ring may also be an integral part of the collimator structure. In any of the aspects, the aiming ring may extend substantially perpendicularly to the longitudinal axis of the elongated arm. In other embodiments, there may be a connecting body component in lieu of an aiming ring which may attach to both the receptor holder and to the collimator. The connecting body portion may also be an integral part of the collimator structure or the receptor holding device. An aiming ring may also be a detachable portion of the connecting body component.

The connecting body portion

The receptor holding arm may be of a curve design, for example, as mentioned above, in the general shape of an S-shaped curve, to facilitate arrangement of the film or digital sensor in a line-of sight of the collimator opening or radiation source. This may also be desirable to minimize parts of the receptor holding arm in the line of the radiation, which may, for example, serve to decrease image artifacts and/or distortions.

In one embodiment, the arm may be ribbon-like, with a width that is larger than its thickness and a substantially flat portion serving as the bite portion. Adjacent the bite portion is a receptor holder that may extend from one end of the elongated arm in a substantially perpendicular upward direction to the flat bite portion. The bite portion may be slightly lower than the mid-section of the receptor holder and the elongated arm may curve upwards gradually to the collimator connection portion. In one aspect, the arm may have an abrupt step down to the bite portion from the lower part of the receptor holder. In another aspect, the arm may have a gradual step down to the bite portion from the lower part of the receptor holder.

In another embodiment, the arm may be in the shape of a rod with a bite portion having a substantially flat surface and a receptor holder that may extend from one end of the arm in a substantially perpendicular upward direction to the flat bite portion. The rod may be of a cross section that is substantially circular, substantially oval, substantially rectangular, substantially triangular, or substantially square. In one aspect, the arm may have an abrupt step down to the bite portion from the lower part of the receptor holder. In another aspect, the arm may have a gradual step down to the bite portion from the lower part of the receptor holder.

In any of the embodiments, the arm may be substantially structurally rigid and has minimal flexing or distortion during operation. This may generally be desirable to ensure proper alignment as flexion may cause misorientation of components of the system.

The receptor holding device may come in different versions or variations, as noted above, for example, for imaging the anterior teeth, posterior or a panoramic view of the oral cavity. X-ray imaging is also carried out during root canal procedures and holding devices may be utilized as well, to follow the progress of the endodontic files, so as to make sure that the endodontic file is going in the correct direction. In general, the holding device may include fins located on and/or near the receptor holder. As a portion of the endodontic file generally sticks out of the tooth undergoing the procedure, the fins may serve as spacers between the two arches of teeth, such as, for example, to keep the two arches separated by a given distance, such as by the gap between the fins, so the file is not in contact with opposing teeth. The fins may vary in dimension and orientation to, for example, accommodate different size files, different depths of file insertion in the tooth, angle or orientation of the file or tooth, different sized sensors or x-ray film, and/or any other variations in endodontic procedures. In some embodiments, the fins may be positioned on said receptor holding device such that they do not substantially impinge on the active area of a receptor. In any of the above embodiments, the s-shaped curve shape of the arm may also be of different shape and with varying location on the curve, depending also on whether the device may be used for imaging the anterior teeth, posterior or a panoramic view of the oral cavity, for example. In addition, different receptor holding devices may also be used to accommodate different sizes of the patients and hence, different curves or locations of the curves.

In one aspect of the invention, the curve shapes of the various receptors holding devices are designed to present an unobstructed path for radiation to travel from the emitter to the film or digital sensors. The receptor holder also provides an unobstructed path for the radiation from the emitter to the film or digital sensor. In other words, the radiation does not pass through any portion of the body of the receptor holder, or the area of the body that radiation passes through is minimized. Though the receptor holder may be constructed of a material not to interfere, distort or attenuate any radiation passing through it, some interference, distortion or attenuation, though minimal, may still occur with any material of a thickness to be structurally rigid, and such interference, distortion or attenuation, may result in artifacts in the final image. In general, the receptor holder may be designed to grasp non-active areas of the receptor, such as portions that are not radiation-receiving. In one embodiment, the receptor holder may be similar to a picture frame with a central aperture where the entire active area of the receptor is unobstructed from the path of the radiation. The frame may include a main structure having claw-like features extending from the main structure of the frame located at various locations of the frame for removably grapping the receptor and retained it in place without extending into the active area of the receptor. In another embodiment, the receptor holder may be similar to a picture frame with a central aperture and the receptor may be removably retained adhesively about the borders of the receptor in the receptor holder. In one aspect, the adhesive may be coated on the receptor holder. In another aspect, the adhesive may be coated on the edges of the sensor away from the active area and attachable to the receptor holder. In a further aspect, the receptor may include a thin sheath enveloping the sensor or film and the adhesive may be coated on the sheath. The sheath may or may not cover the active area of the receptor. When the sheath also covers the active area of the receptor, it may be made of a very thin material or the material may be of a type that minimizes any possible creation of artifacts in the imaging process, as structure property is not as important for the sheath as for parts of the holder itself. According to one embodiment, the sheath may be reusable and adhesively attached to the receptor holder and the receptor may be removably disposed inside the sheath during use. According to another embodiment, the sheath may be part of the receptor assembly and is not reusable.

The adhesive may be applied either in the manufacturing process or by the user, such as by the dental practitioner or his assistant. The adhesive may be applied to any of the embodiments described above by any known coating method, such as spray on, or dip-coat, during manufacturing, or applied on any of the surface as a doubled-coated adhesive strip. The adhesive may be any pressure sensitive which may or may not be removable or repositionable, may be a hot melt pressure adhesive which may or may not be removable or repositionable.

Though the receptor holder may either be separate from the rest of the receptor holding device, namely, the elongated arm, the one piece design of integrally formed receptor holder and elongated arm presents several advantages including proper alignment of the holder without any need for adjustment, fewer parts to assemble during operation, and faster, therefore, less time consuming for the patient and practitioner and thus less discomfort for the patient.

Though using a different receptor holding device for different teeth also has advantages, including proper alignment without adjustment while the patient is waiting, fewer chances of misalignment leading to retakes, and time saving in general, an adjustable or modular holding device may also accomplish the same function with complementary features for connection.

The connection between the receptor holding device and the collimator may be effected in various manners. For example, the connection may be effected with complementary features at the connection portion of the elongated arm and the outside of the collimator. The connection may also be between the receptor holder to a separate aiming ring, which may then also connect to the collimator. This may be desirable such that the receptor holder and aiming ring may be utilized without directly coupling to a collimator, such as, for example, when utilizing unusual alignments or angles, or with other collimators and/or no collimator at all. Also, a separate aiming ring which couples to both the receptor holder and to the collimator may be desirable, as distance adjustments can be made with the aiming ring attached to the receptor holder prior to attaching to the collimator, which is stationary on the radiation source and more difficult to move. The aiming ring itself may also include magnetic elements which may, for example, be used to couple magnetically directly to an appropriate metal x-ray tube.

In one embodiment, the connection may be effected with magnetic force. According to one embodiment, a magnetic element may be permanently attached to the connection portion of the receptor holder and a complementary magnetic element may be present on the outside of the collimator. According to another embodiment, the magnetic element may be present on a separate part that may be removably attached to the connection portion of the arm. Also, the magnetic elements may be beneath the actual surface of the collimator and/or the receptor holder, as the magnetic force may penetrate the material of the components and still function. This may be desirable for cosmetic reasons and also to accommodate other features, such as guiding and/or alignment features.

In one aspect, the connection portion may include a depression for seating the magnetic element so that the magnetic element barely protrudes above the profile of the elongated arm. In another aspect, the magnetic element may be seated on the surface of the elongated arm so that it protrudes above the profile of the arm. The magnetic element may also be concealed beneath the surface of the elongated arm.

The complementary magnetic element on the outside of the collimator may include multiple connecting points, for accommodating the various receptor holding devices to keep the sensor centered in the aperture or opening of the collimator.

In addition to making a proper and secure connection, the magnetic connection may also be self guiding. When the complementary components are in the vicinity, they may guide each to the other to make the proper connection as generally the magnetic materials or elements will align at or close to the configuration of maximum attractive force. The magnetic strength of either part may be strong enough for a secure connection while easily removable at the same time. Removability may usually be affected by a twisting or torque action. External aids such as an additional locking mechanism may or may generally not be needed with magnetic connection. Thus, the magnetic attachment mechanism between the receptor holding device and the collimator not only provides a secure and accurate attachment, it also gives the added ability to guide the receptor holding device to the proper attachment location on the collimator, further aids in facilitating the imaging process.

In another embodiment, the complementary parts may be a tab, a raised part or protrusion on one component and a properly sized cavity or channel for the tab, raised part or protrusion to be seated firmly, either by friction or with external aids, such as an adhesive or a locking mechanism.

In general, the magnetic elements may also be used to complement physical interlocking and/or complementary structures on the collimator and the receptor holder.

In other embodiments, the complementary parts may be nesting such that any action of the magnetic forces of the magnetic materials or elements may generally result in one part nesting into the other in a predicted fashion. This may be desirable as it may serve to minimize or eliminate chances of user error. The nesting parts may also be designed such that only one nesting configuration is possible, which may generally be the proper configuration.

Also, the connection features may also be as large as possible to facilitate a more secure connection during use without being obtrusive, as noted before.

The connecting body portions may also include handle portions, for example, a pair of handle portions, to facilitate handling and connection. The handle portions may be of any sizes and orientation as long as they do not obstruct the line of sight. In one example, the handle portions maybe similar to rabbit ears and include grip portions for easy handling or gripping. The grip portions may also be at least one of roughened, having a depression, and coated with higher friction material to facilitate gripping.

When the aiming ring is used, the aiming ring may also include handle portions, for example, a pair of handle portions, to facilitate handling and connection, similar to those on the connecting body portion alone, without the ring portion.

With high sensitivity sensors, filters may be used to decrease the radiation dosage needed for imaging. Filters maybe part of or attached to the aiming ring, if used.

For example, sensors using scintillation technology may enable high resolution image capture with increased contrast ratio, resulting in more diagnostic detail in the radiograph. For another example, CMOS image detector chip with 18 bit image capture may also result in higher resolution and reduced pixilation when zooming in on small details.

Sensors may also include ergonomically designed body with rounded corners and edges without clipping the corners of the active area, resulting in a more comfortable sensor without the loss of anatomical coverage.

In general, all the parts of the system cooperate to facilitate the imaging process to create an image that is representative of the actual object with few artifacts.

The present invention together with the above and other advantages may best be understood from the following detailed description of the embodiments of the invention illustrated in the drawings below.

### BRIEF DESCRIPTION OF THE DRAWINGS

FIG. 1 illustrates an exploded view of an embodiment of an imaging and alignment system of the present invention;

FIG. 1A shows a perspective view of an assembled imaging and alignment system of FIG. 1 in an embodiment of the invention;

FIGS. 1B, 1C, 1D and 1E illustrate perspective views of an embodiment of a collimator and aiming ring with enlarged complementary mountings and handles;

FIGS. 2, 2A, 2B and 2C illustrate a front perspective view, a back perspective view, a front view and a back view, respectively, of an embodiment of a collimator with an internal attachment mechanism;

FIG. 2D illustrates a collimator with multiple mountings for an aiming ring or a connecting body;

FIGS. 2E-K illustrate the front perspective view, front view, back view, right side view, top view, bottom view and left side view, respectively, of a collimator with enlarged mountings for an aiming ring or a connecting body;

FIGS. 2L, and 2L-1 to 2L-6 illustrate the front perspective view, front view, back view, right side view, top view, left side view, and bottom view, respectively, of a collimator with enlarged mountings coupled with an aiming ring with handles coupled to a receptor holding device coupled to a receptor;

FIGS. 3 and 3A illustrate detailed perspective views of the attachment mechanism in an embodiment of the present invention;

FIGS. 3B and C illustrates an embodiment of the attachment arms in a closed and open position, respectively, of an internal attachment mechanism of an embodiment of the present invention;

FIG. 3D shows an exploded view of the plates in an embodiment of the internal attachment mechanism of the present invention;

FIG. 3E illustrates the internal attachment mechanism engaging the inner surface of an x-ray tube;

FIG. 4 illustrates a front perspective view of an embodiment of an external attachment mechanism of the present invention;

FIG. 4A illustrates a back perspective view of an embodiment of an external attachment mechanism of the present invention;

FIG. 4B illustrates the back view of an embodiment of an external attachment mechanism of the present invention;

FIG. 4C illustrates an embodiment of an external attachment mechanism of the present invention, showing the attachment to a radiation source;

FIGS. 4D and 4E illustrate an iris-like attachment mechanism;

FIGS. 4F and 4G illustrate a ribbon-like attachment mechanism;

FIG. 4H illustrates a telescoping attachment mechanism;

FIG. 5 illustrates an embodiment of an aperture control mechanism;

FIG. 5A shows a detailed view of an embodiment of an aperture control mechanism;

FIGS. 5B and 5C illustrate portions of a multiple plate aperture control mechanism;

FIG. 5D illustrates the interaction of an aperture control handle with a plate of a multiple plate aperture control mechanism;

FIGS. 6 and 6E show front and rear perspective views, respectively, of an aiming ring in one embodiment of the present invention;

FIG. 6A illustrates the interaction of an aiming ring with a receptor holder;

FIG. 6B illustrates a connecting body in an embodiment of the present invention;

FIG. 6C illustrates an embodiment of a collimator with mounting features in one embodiment of the present invention;

FIG. 6D illustrates an embodiment of a detachable aiming ring with multiple mounting features;

FIGS. 6F and 6G illustrate the front and back perspective views, respectively, of a connecting body with handles and an enlarged mounting portion;

FIGS. 6H and 6H-1 illustrate attachment of a receptor holding device via an aiming ring to the collimator at a horizontal and vertical orientation, respectively, via different mounting portions on the collimator;

FIGS. 6I and 6I-1 to 6I-7 illustrate the front perspective view, top view, bottom view, rear perspective view, front view, back view, left side view and right side view, respectively, of an aiming ring with handles and an enlarged mounting portion;

FIGS. 7 and 7A show back and front perspective views, respectively, of a receptor holding device with a crossbar in an embodiment of the present invention;

FIG. 7B illustrates multiple embodiments of receptor holding devices;

FIGS. 7C and 7D show front and back perspective views, respectively, of a receptor holding device without a crossbar in an embodiment of the present invention;

FIGS. 7E and 7E-1 to 7E-7 illustrate the back perspective view, front perspective view, front view, rear view, left side view, top view, right side view and bottom view, respectively, of a horizontal middle receptor holding device of an embodiment of the present invention;

FIGS. 7F and 7F-1 to 7F-7 illustrate the front perspective view, rear perspective view, front view, rear view, left side view, bottom view, right side view and top view, respectively, of a horizontal left receptor holding device of an embodiment of the present invention;

FIGS. 7G and 7G-1 to 7G-7 illustrate the front perspective view, rear perspective view, front view, rear view, left side view, right side view, top view and bottom view, respectively, of a horizontal right receptor holding device of an embodiment of the present invention;

FIGS. 7H and 7H-1 to 7H-7 illustrate the rear perspective view, front perspective view, front view, rear view, top view, bottom view, right side view and left side view, respectively, of a vertical middle receptor holding device of an embodiment of the present invention;

FIGS. 7I and 7I-1 to 7I-7 illustrate the rear perspective view, front perspective view, front view, rear view, top view, bottom view, right side view and left side view, respectively, of an anterior receptor holding device of an embodiment of the present invention;

FIGS. 7J and 7J-1 to 7J-7 illustrate the front perspective view, rear perspective view, front view, rear view, left side view, right side view, top view and bottom view, respectively, of an endodontic receptor holding device of an embodiment of the present invention;

FIGS. 7K and 7K-1 to 7K-7 illustrate the front perspective view, rear perspective view, front view, rear view, left side view, right side view, top view and bottom view, respectively, of an endodontic receptor holding device of an embodiment of the present invention;

FIGS. 8 and 8A illustrate a multiple piece receptor holding device and its assembly;

FIGS. 8A-1 to 8A-5 illustrate holder portions of a multiple piece receptor holding device to form a horizontal middle, horizontal left, horizontal right, vertical middle and an anterior receptor holding device, respectively;

FIGS. 8B and 8B-1 illustrate a dropped arm portion of a multiple piece receptor holding device;

FIGS. 8C and 8C-1 illustrate a raised arm portion of a multiple piece receptor holding device; and

FIGS. 8D and 8D-1 illustrate a no-rise arm portion of a multiple piece receptor holding device;

FIGS. 9, 9A and 9B illustrate an x-ray receptor; and

FIG. 9C illustrates an interface box for use with an x-ray receptor.

### DETAILED DESCRIPTION OF THE INVENTION

The detailed description set forth below is intended as a description of the presently exemplified methods, devices and system provided in accordance with aspects of the present invention and is not intended to represent the only forms of the present invention. It is to be understood, however, that the same or equivalent functions and components incorporated in the methods, devices and system may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

The present invention relates to a system, method and device for capturing dental and/or medical images. The images may be captured on and/or by a receptor which may include, for example, film or an electronic sensor such as a digital sensor or a camera. The system includes a positioning and aiming device with improved connection features between a collimator structure and an emitter tube as well as between a collimator structure and a receptor holder, creating an unobstructed line of sight between the collimator aperture and the receptor. The collimator structure may include an aiming ring that may be integral or separate from the collimator structure with improved attachment features. The components all cooperate to produce dental and/or medical images with improved aiming and/or alignment, and requiring minimal assembly during and/or just prior to actual operation to acquire images. The system, method and device are capable of producing better quality images, reducing retakes, and/or reducing artifacts, thus minimizing exposure of patients to radiation unnecessarily than the operation with any conventional radiation source already in use.

The present invention also relates to integrated receptor holding devices that minimize assembly just before use to improve efficiency and faster and better alignment; or interchangeable parts, all with improved attachment features cooperating to facilitate operation.

The present invention further relates to a system having above mentioned improvements to facilitate operation.

As mentioned above, in medical imaging, there are various ways to capture images of patients, for example, for diagnostic purposes. A medical professional such as a dentist may use a dental X-ray device to capture on a receptor, such as film or electronic sensor, an X-ray image of a patient's mouth. An electronic sensor may generally be used to capture digital image and generally may include a computer workstation, in addition to the electronic sensor, associated with the X-ray device. Digital cameras may also a form of electronic sensor and be used to capture still and video images for later storage on a computer in the patient record.

A typical X-ray imaging system in its simplest form may include the following components: an X-ray device having a generator or emitter tube where X-rays are generated; a collimator, which may act as a collimator in the traditional sense or may act more as a shield than a true collimator in the traditional sense of collimation, and may be in different forms, for example, tube-form or plate form, having an aperture, which may have varying shapes; and a receptor to capture the image. An aiming device to aid in aiming the X-ray device to avoid unnecessary exposure to the patient and possible repeatability of the operation may or may not be needed, depending on whether this function may be adequately performed by other components already present, as note below. As each of the components mentioned above are typically separate components and typically may act independently, proper integration of the components to act as a system is important in helping to reduce artifacts, potential patient exposure to unnecessary radiation, improve focus, reproducibility or ease of operation. Also, in some imaging processes, the aiming ring may be used without the collimator.

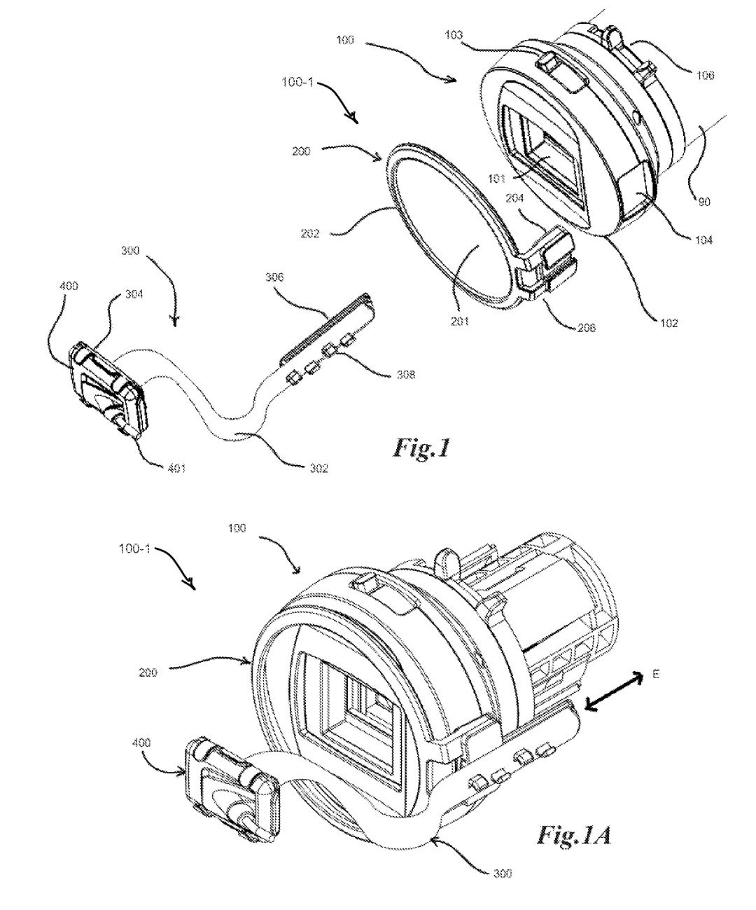

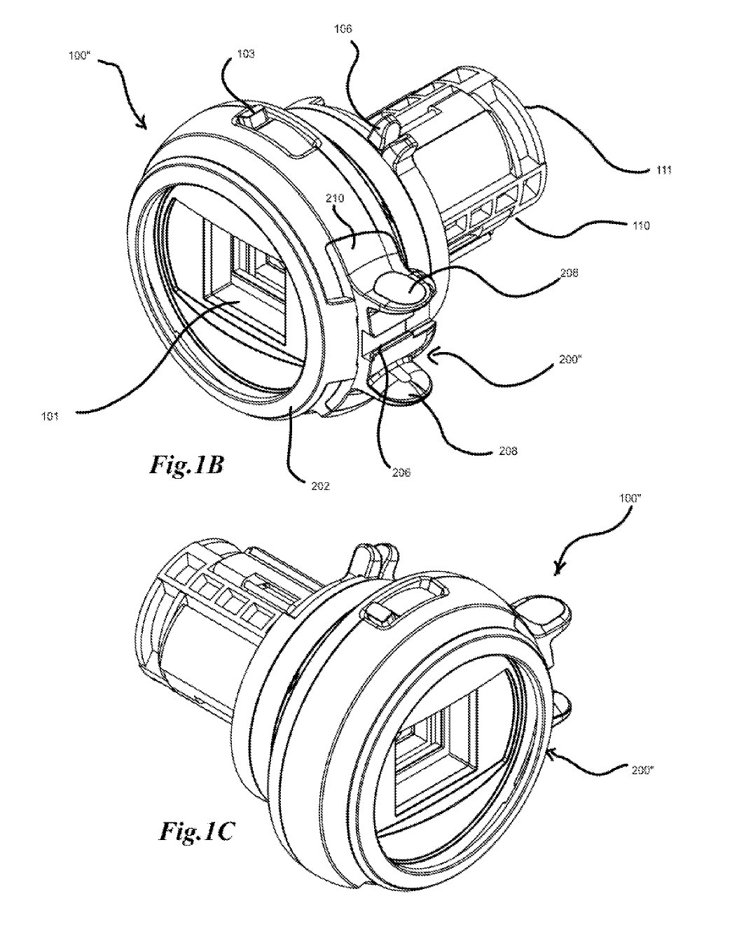

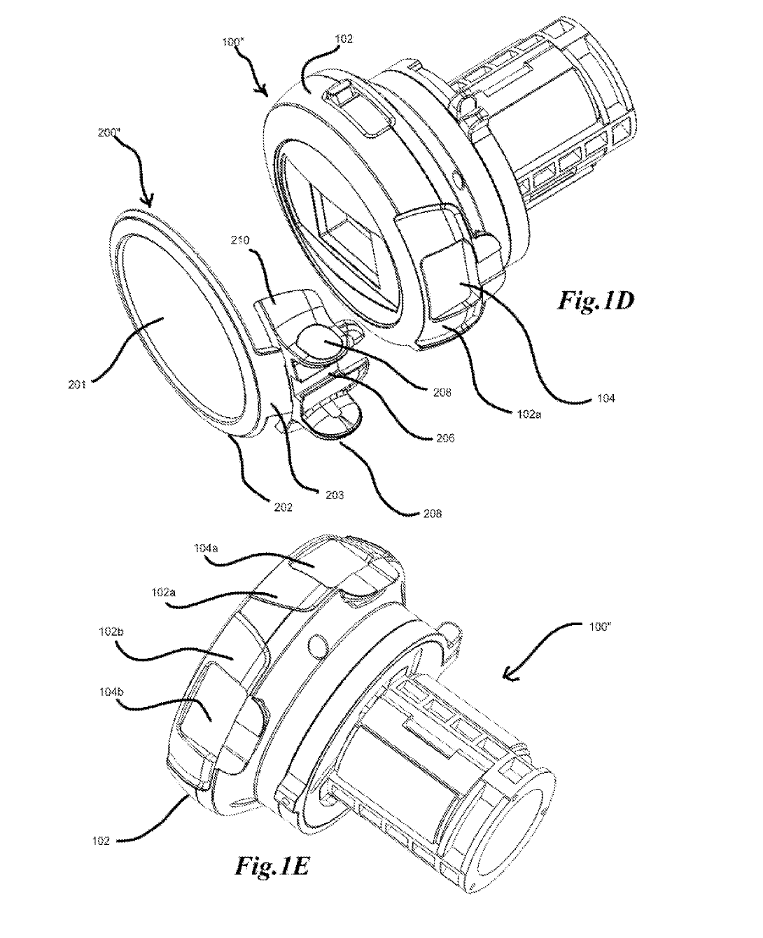

FIG. 1 illustrates an embodiment of an imaging and alignment system which may generally include a collimator structure 100-1 which attaches to a radiation source, such as the output or emitter tube 90 of a dental x-ray, an aiming ring 200, a receptor holding device 300, and a receptor 400. The collimator structure 100-1 may include a collimator 100 with an opening or aperture 101, for collimation of, or shielding or shaping of, for example, an X-ray beam to conform more accurately to the size and shape of the receptor 400, such as an x-ray film or electronic sensor, held by the receptor holder 300 so as to minimize excess radiation exposure. The collimator 100 may in general, be a separate, detachable device connecting to the emitter tube 90 and the receptor holding device 300.

In general operation, radiation from the source, such as the output tube 90, may pass through an aperture 101 in the collimator 100, optionally through the aiming ring 200, if present, and incident on the receptor 400 which is held in a fixed position relative to the collimator 100 by the receptor holding device 300, as illustrated with the components in a fully assembled and attached configuration in FIG. 1A.

In another embodiment, an assembled configuration, as shown in various views in

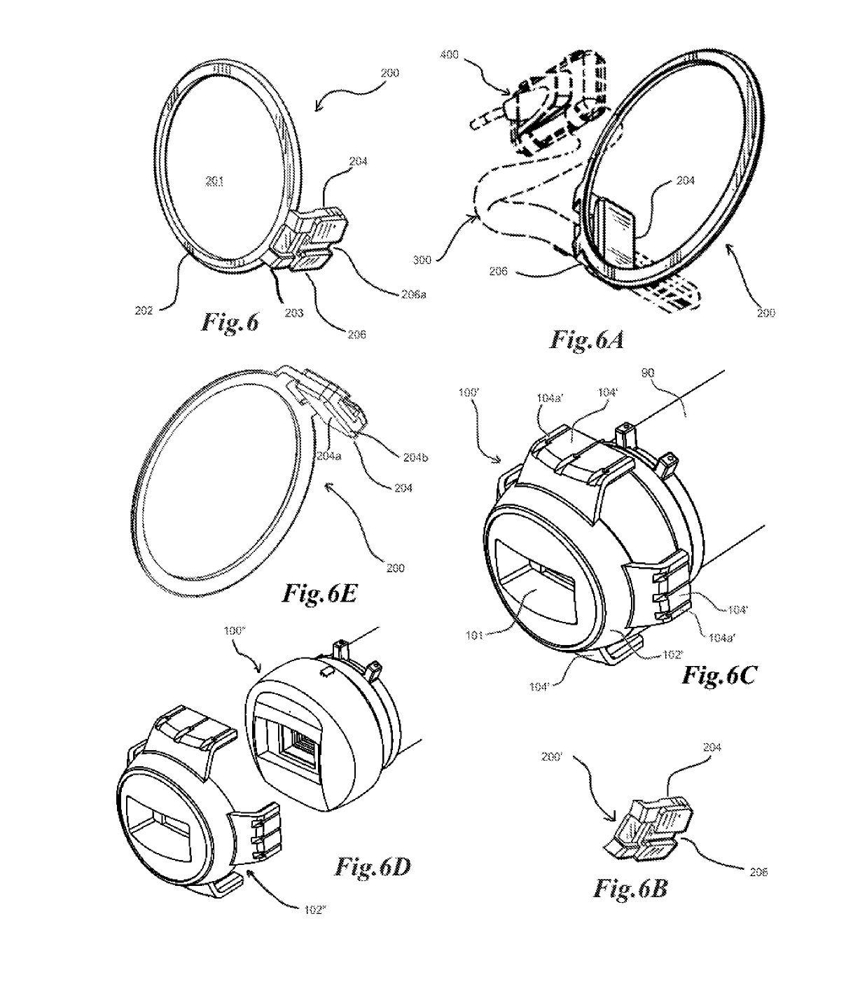

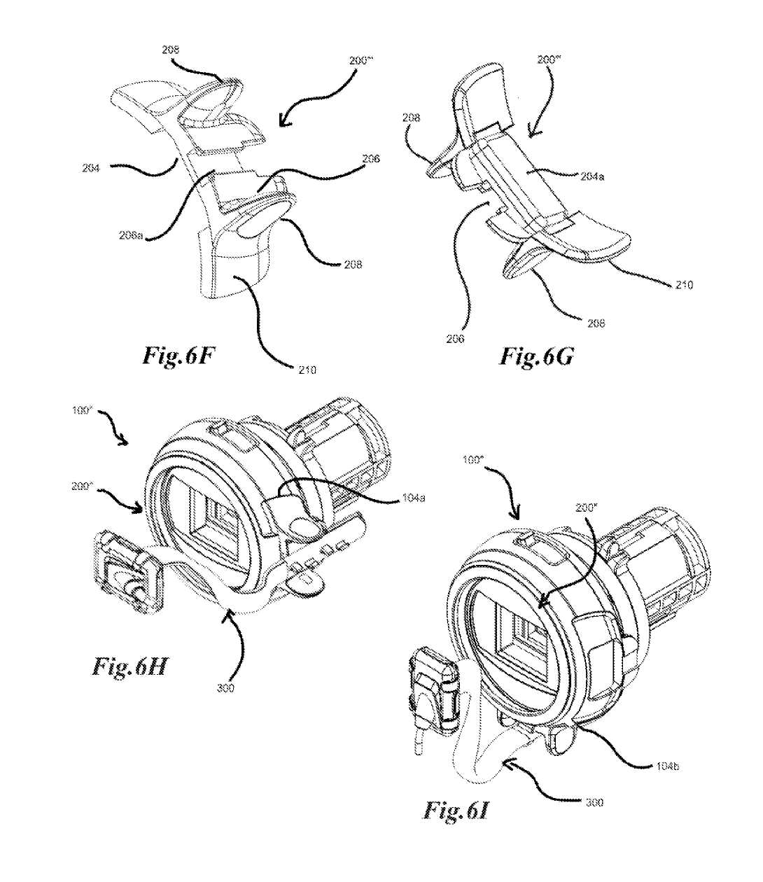

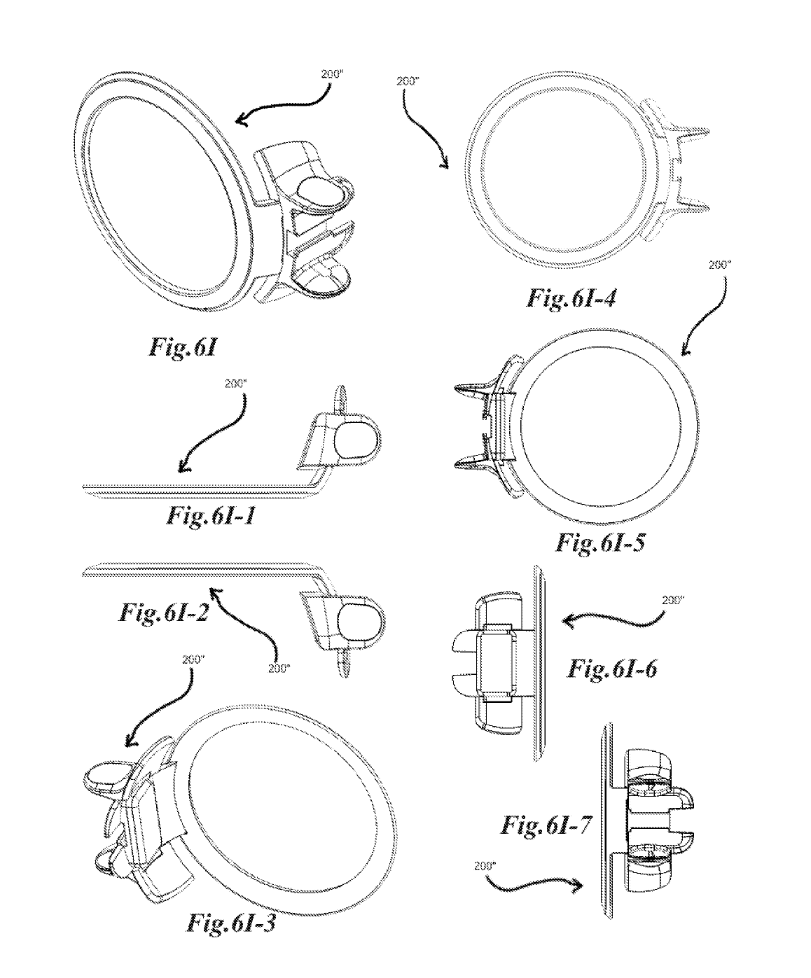

FIGS. 2L and 2L-1 to 2L-6, similar to that of FIG. 1A, except that the aiming ring includes handles, for example 208, as illustrated in FIGS. 1B, 1C, 1D, 6F and 6G, and an enlarged mounting portion with corresponding enlarged stepped mounting depressions on the collimator, for example, 102a and 102b, is shown in FIGS. 1D and 1E.

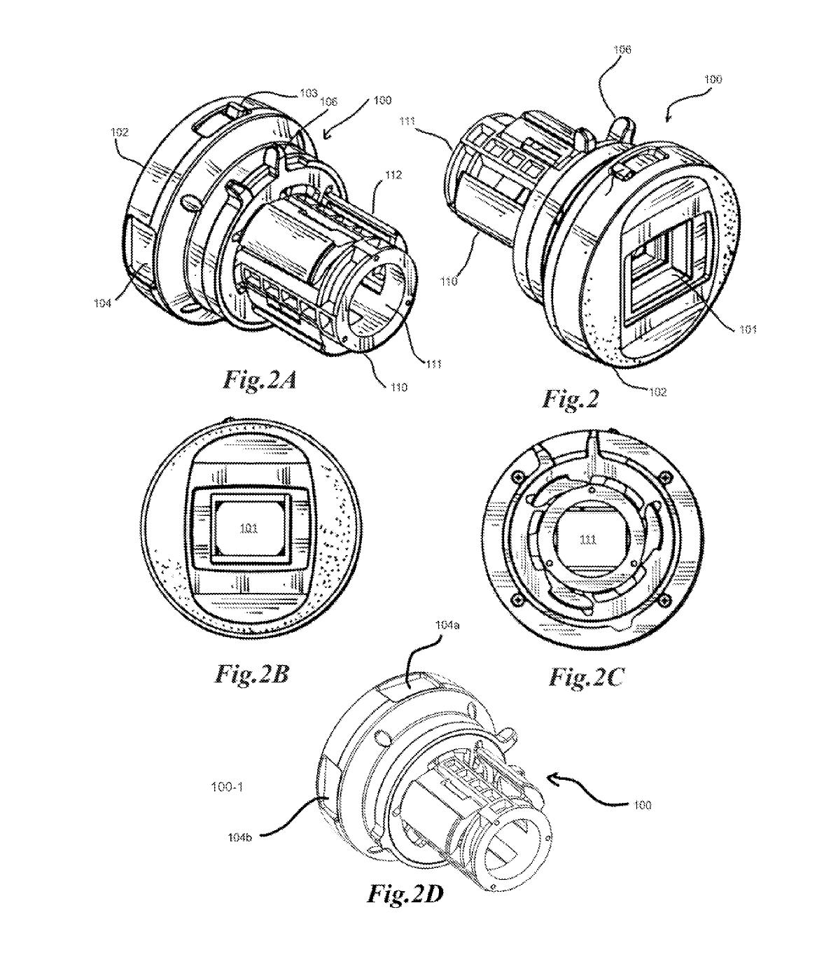

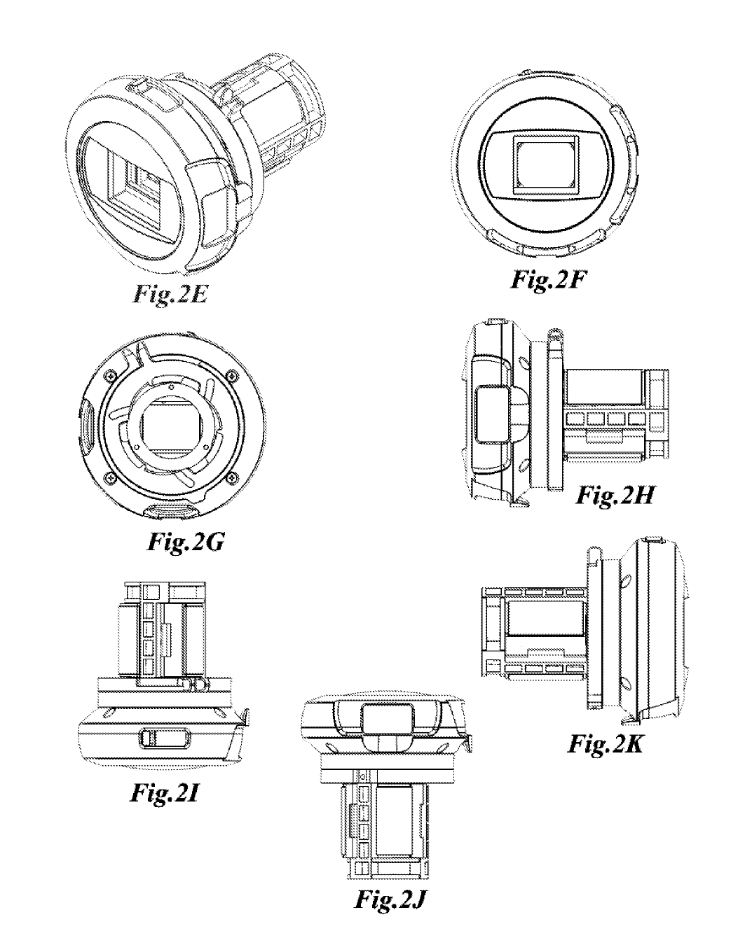

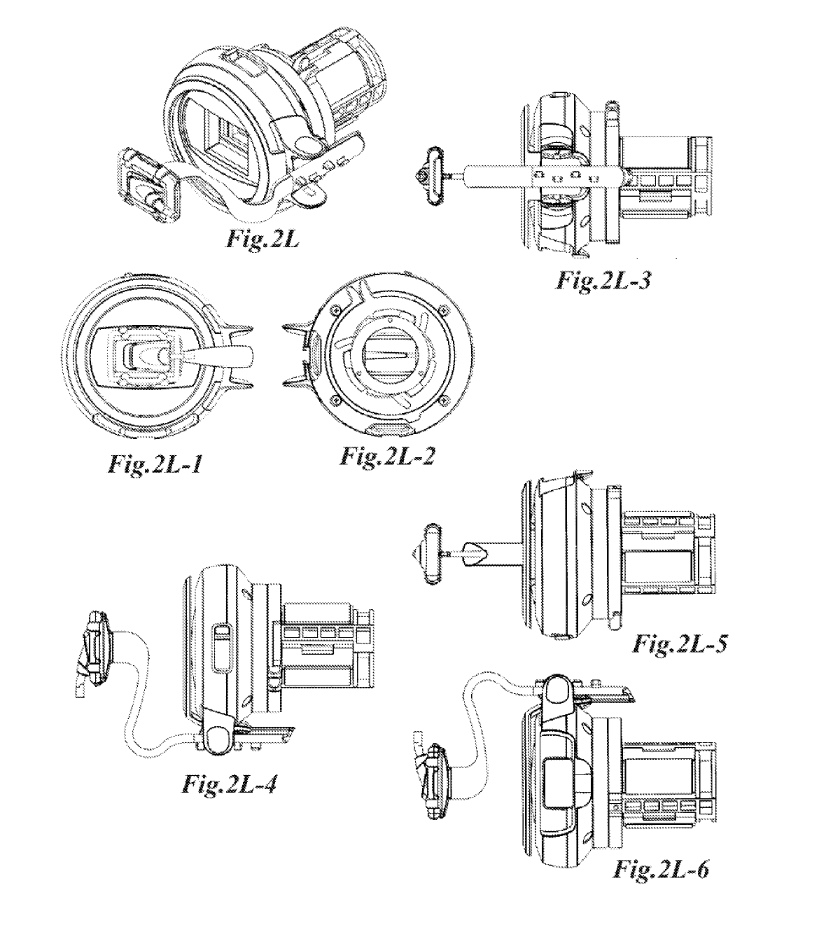

The shape of the collimator opening or aperture 101 and its attachment to the X-ray generator emitter tube 90 may influence the operation of the imaging device. Traditional collimators present a circular aperture to the patient. More recently, a collimator 100, as shown in FIGS. 1 and 1A, having a rectangular aperture is found to reduce a patient's exposure to unwanted radiation. This rectangular aperture 101 may be either inherent to the collimator 100 itself or by an internal aperture insert or a plate externally attached. Whether the aperture is circular or rectangular in shape, proper attachment of the collimator 100 to the generator or emitter tube 90 and proper alignment with the receptor holder to produce an unobstructed line of sight between the aperture 101 and the receptor 400, as shown in FIG. 1A, or the different embodiment of FIG. 2L, all may contribute to reduce artifacts, potential patient exposure to unnecessary radiation, improve focus, reproducibility or ease of operation.

The main frame 102 of the collimator 100 itself may be substantially cylindrical, as shown in FIG. 1. In one aspect, the frame may have a small length, for example, in the form of a thick plate with a central opening. In another aspect, the collimator may have some length defining a short tubular structure.

In one embodiment, as illustrated, for example, in FIGS. 2 and 2B, a collimator 100 may generally include a main body 102 with an aperture 101 through which radiation from a radiation source may exit the collimator 100. The collimator 100 may further generally include an attachment mechanism 110 with a further aperture 111 which allows radiation from a radiation source to enter the collimator 100 before exiting through aperture 101 in the main body 102, as shown for example, in FIGS. 2A and 2C. The collimator 100 may also generally include handling and/or adjustment features to, for example, control the size and/or shape of the aperture 101 and to control the attachment mechanism 110, such as with aperture control 103 and attachment control 106, respectively. The collimator 100 may further include a mounting feature 104, as shown for example, in FIG. 2A, on the main body 102 for attachment and/or coupling of the collimator 100 to other components, such as the aiming ring 200 if present, or a receptor holder 300, as shown, for example, in FIGS. 1, and 1A.

The collimator structure 100-1 may be constructed of metal, metallic material, and/or polymeric material doped with radiation blocking, or absorbing material, such as metal or metal oxides. Examples of suitable metals or metallic materials may include lead, tin or similar. Examples of dopants for polymeric materials may include lead and Bismuth oxides.

The collimator structure 100-1 may also be fabricated from any metallic and polymeric materials, as long as such material may be fabricated into the resulting rigid or substantially rigid parts. Examples of appropriate materials may include, but are not limited to, for example, a polymer that may be molded, thermoformed or cast. Suitable polymers include polyethylene; polypropylene; polybutylene; polystyrene; polyester; polytetrafluoroethylene (PTFE); acrylic polymers; polyvinylchloride; Acetal polymers such as polyoxymethylene or Delrin (available from DuPont Company); natural or synthetic rubber; polyamide, or other high temperature polymers such as polyetherimide like ULTEM®, a polymeric alloy such as Xenoy® resin, which is a composite of polycarbonate and polybutyleneterephthalate, Lexan® plastic, which is a copolymer of polycarbonate and isophthalate terephthalate resorcinol resin (all available from GE Plastics); liquid crystal polymers, such as an aromatic polyester or an aromatic polyester amide containing, as a constituent, at least one compound selected from the group consisting of an aromatic hydroxycarboxylic acid (such as hydroxybenzoate (rigid monomer), hydroxynaphthoate (flexible monomer), an aromatic hydroxyamine and an aromatic diamine, (exemplified in U.S. Pat. Nos. 6,242,063, 6,274,242, 6,643,552 and 6,797,198, the contents of which are incorporated herein by reference), polyesterimide anhydrides with terminal anhydride group or lateral anhydrides (exemplified in U.S. Pat. No. 6,730,377, the content of which is incorporated herein by reference)or combinations thereof Some of these materials are recyclable or be made to be recyclable. Compostable or biodegradable materials may also be used and may include any biodegradable or biocompostable polyesters such as a polylactic acid resin (comprising L-lactic acid and D-lactic acid) and polyglycolic acid (PGA), polyhydroxyvalerate/hydroxybutyrate resin (PHBV) (copolymer of 3-hydroxy butyric acid and 3-hydroxy pentanoic acid (3-hydroxy valeric acid) and polyhydroxyalkanoate (PHA) copolymers, and polyester/urethane resin. Some non-compostable or non-biodegradable materials may also be made compostable or biodegradable by the addition of certain additives, for example, any oxo-biodegradable additive such as D2W™ supplied by (Symphony Environmental, Borehamwood, United Kingdom) and TDPA® manufactured by EPI Environmental Products Inc. Vancouver, British Columbia, Canada.

In addition, any polymeric composite such as engineering prepregs or composites, which are polymers filled with pigments, carbon particles, silica, glass fibers, or mixtures thereof may also be used. For example, a blend of polycarbonate and ABS (Acrylonitrile Butadiene Styrene) may be used for the housing 132 and sleeve 108. For further example, carbon-fiber and/or glass-fiber reinforced plastic may also be used.

Useful metals or metallic materials may include metal and metal alloys such as aluminum, steel, stainless steel, nickel titanium alloys and so on.

Some materials possess higher rigidity that others and therefore thinner components may have sufficient rigidity. For those that are more flexible, thicker components may be needed to provide sufficient rigidity.

The collimator structure 100-1 may be fabricated from the same material or different materials, such as those mentioned above.

To accommodate different size radiation emitter tubes 90 such as the X-ray emitter tubes 90, and to ensure secure retention of the collimator structure 100-1 to the radiation source, such as the X-ray emitter tube 90, the collimator structure 100-1 may include an attachment mechanism 110, which may include either an external or internal attachment, either adjustable to fit all sizes of emitter tubes or specific to one particular emitter tube size.

In some embodiments, the attachment mechanism 110 may be utilized to insert into the output tube of a radiation source, such as the x-ray output tube 90 in FIG. 1. FIGS. 1B and 2A shows an embodiment of an internal attachment mechanism 110 which may generally include a plurality of arms 112 which may be used to engage the inner surface of the output tube 90 to hold the collimator 100 in place. The engagement surface may generally have a high frictional surface that may include friction-enhancing features, such as being constructed from a high frictional material, having a coating of high frictional material, and/or having an attachment of a high frictional material such that the surface that touches the inside of the radiation source may have enhanced frictional engagement, as noted before.

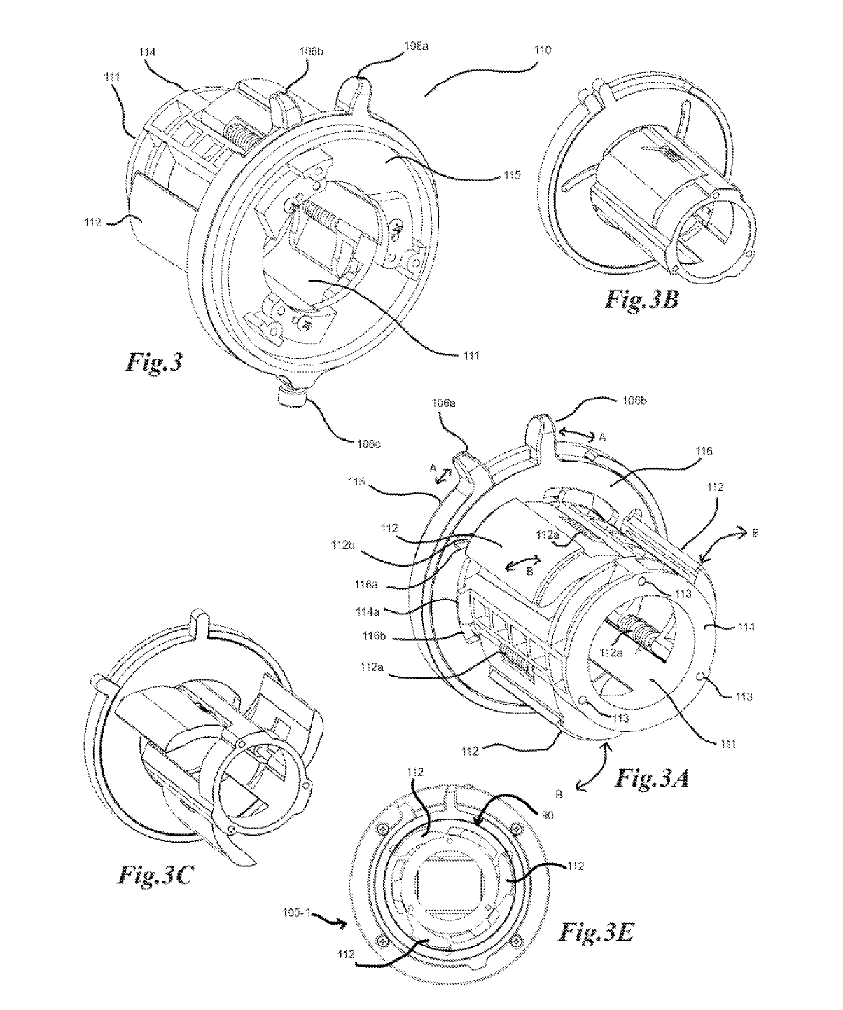

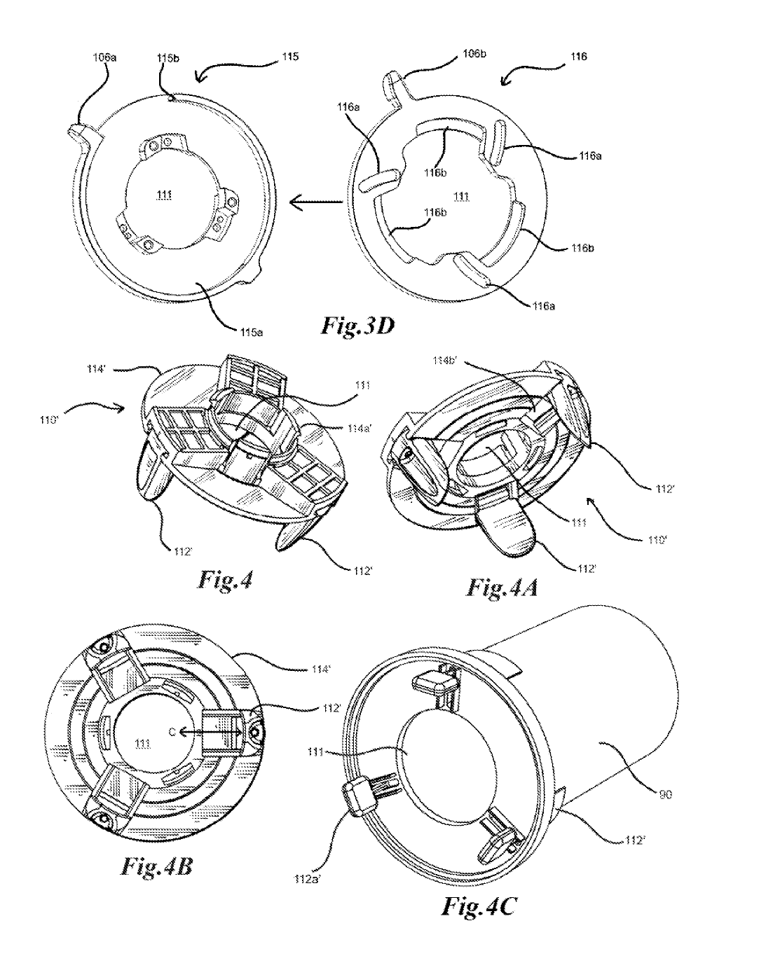

To accommodate different size radiation emitter tubes 90 and to ensure secure retention of the collimator 100 to the tube 90, the attachment mechanism 110 as illustrated in FIG. 3, may be envisioned. This embodiment of the internal attachment mechanism 110 may be adapted for attachment to various sizes emitter tubes 90 with substantially the same components, cooperating to effect a secure attachment.

The connection mechanism 110 features an internal attachment mechanism 110, the cooperating components of which are as illustrated in FIG. 3 includes expanding arms 112 which are actuated to engage the inner surface of the emitter tube (not shown). The arms 112 may generally be made of a rigid or substantially rigid material to, for example, aid in secure frictional engagement to the inner surface of the tube 90. Less rigid materials may entail larger thickness.

The arms 112 may, as illustrated in FIGS. 3 and 3A, pivot on fixed pivot points 113 and expand radially in a direction B from a central cylindrical frame 114, through which there is an aperture 111 for radiation, such as x-rays, to pass through. In one embodiment, the non-pivotal ends of the arms 112 further include pins 112b which rest within radially arcuate slots 116a in a plate 116, which is rotationally coupled against a second plate 115, which is also illustrated in an exploded view of the plates 115, 116 in FIG. 3D. The plate 116 may also generally nest into the plate 115 in a depression 115a, as shown in FIG. 3D, with the rim 115b acting as a rotational stop for the handle 106b. Handles 106a and 106b of plates 115 and 116, respectively, are used to rotate the plates in opposite angular directions by pushing toward each other along direction A. The rotation of the plates 115, 116 causes the pins 112b of arms 112 to translate within the slots 116a, which in turn draws the non-pivotal ends of the arms 112 radially inward or outward from the central cylindrical frame 114 in direction B. The cylindrical frame 114 may also rotate during the movement of the arms 112, such as by translating tabs 114a within arcuate slots 116b of plate 116 as shown in FIG. 3A. The arms 112 may then be utilized to engage the inner surface of the x-ray tube 90 to provide a frictional engagement to retain the collimator 100 to the tube 90, as shown with the arms in a fully retracted position in FIG. 3B and in the fully extended position of FIG. 3C. The arms 112 may further include biasing springs or other biasing members 112a, which are illustrated as torsion springs, which may bias the arms 112 in an open configuration (radially outward from central cylindrical frame 114, as illustrated in FIG. 3A), or alternatively in a closed configuration (radially inward toward the central cylindrical frame 114). As illustrated with the biasing members 112a in FIG. 3A, after the handles 106a, 106b are released, the biasing members 112a may provide the biasing force to push the arms 112 outward in direction B to engage the inner surface of the tube 90. To retain the plates 115, 116 in any particular position relative to each other, a set screw 106c and/or other setting or securing feature may also be utilized.

The springs may be metallic or polymeric, and may include any of the above mentioned materials, as long as they provide sufficient biasing forces to effect attachment.

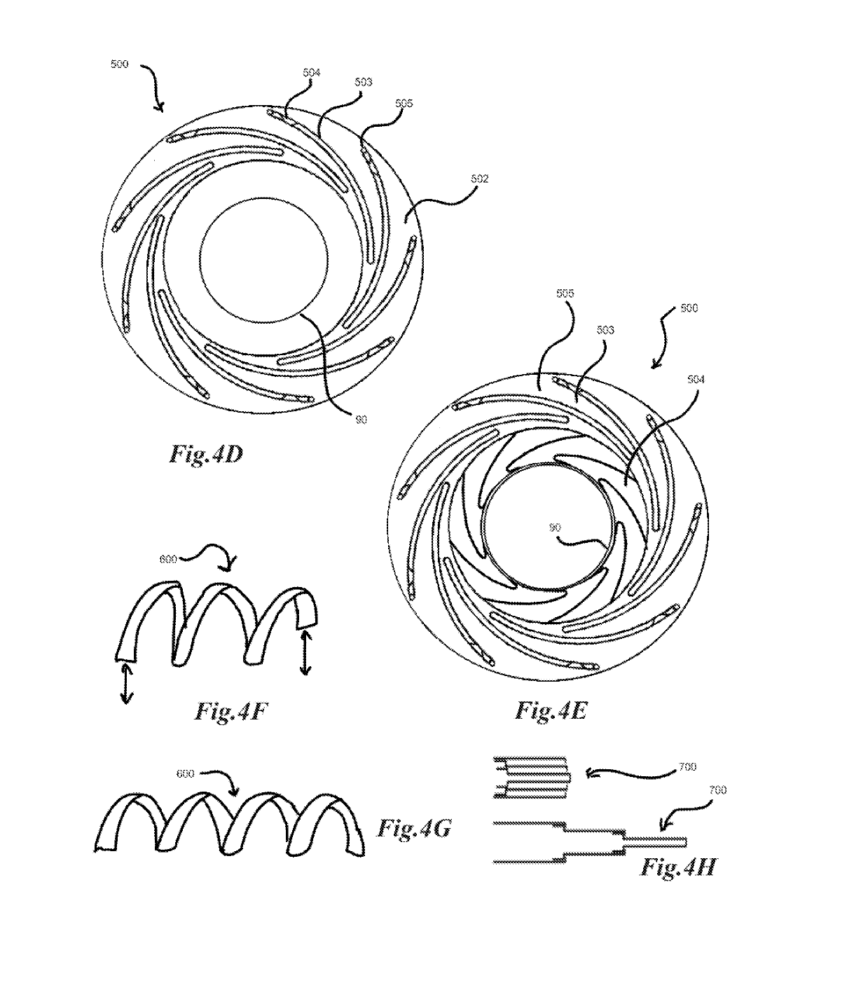

In a further embodiment, an internal connection mechanism, an example of which is illustrated in FIGS. 4D and 4E with mechanism 500, may include a central frame having spirally arranged overlapping portions nested together prior to use and expanded to the proper connection size during use, in a manner similar to an iris. The portions may be contracted, such as by increasing their overlap, to fit the large circumferential emitter tubes and expanded, such as by decreasing their overlap, to fit the smaller circumferential size emitter tubes. The spirally arranged portions may include features to lock the device in place once a proper size is found. FIGS. 4D and 4E illustrate an example of an iris-like mechanism 500 which may generally include a circumferential plate 502 with a plurality of arcuate slots 503 in which pins 505 of a plurality of leafs 504 may travel. As the pins 505 move in the arcuate slots 503, the leafs 504 are moved and the degree of overlap of the leafs 504 may thus vary to change the size of a central opening to fit around an X-ray tube 90, as illustrated.

In yet a further embodiment, the external attachment mechanism, an example of which is illustrated in FIGS. 4F and 4G with ribbon 600, may include a spirally wound device that may expand or contract circumferentially to fit over the size of the emitter tube used and grasp the emitter tube by pressing inward. The spiral may be constructed of a length of ribbon-like material, located on a central frame which may have tracks. By twisting and/or torsioning the ribbon-like material, the spiral expands or contracts in circumferential and/or radial sizes to accommodate the different circumferential size of emitter tubes. This mechanism may also be utilized as an internal attachment mechanism having similar spirally wound components, where the spirally wound device may expand or contract circumferentially to fit the inner size of the emitter tube used and push outward against the inside of the emitter tube. FIGS. 4F and 4G illustrate a ribbon 600 which may be torsioned as shown to vary its dimension to clamp down on the outside of an X-ray tube or to expand into the inside of an X-ray tube, as shown with the decrease in dimension from FIG. 4F to FIG. 4G, or vice versa.

Likewise, the internal attachment mechanism may be constructed of similar materials useful for the collimator, mentioned above, as long as the material may be fabricated into rigid or substantially rigid components, such as the arms 112, the central frame, plates 115 and 116, biasing springs and attachment parts.

In general, the arms 112, for example, as shown in the back view of the collimator 100 inserted into an emitter tube in FIG. 3E, when expanded, may rest against the inner surface of the x-ray tube 90 frictionally, as shown with the arms 112 in contact with the inner surface of the x-ray tube 90. Therefore, any polymeric material listed above that are not capable of having a frictionless surface, or substantially frictionless surface, such as polytetrafluoroethylene (PTFE—Teflon), may be more desirable. For the materials that lack sufficient frictional properties, the surfaces of contact may be roughened or coated with materials having sufficient frictional properties. In general, higher frictional properties include friction-enhancing features, such as being constructed from a high frictional material, having a coating of high frictional material, and/or having an attachment of a high frictional material such that the surface that touches the inside of the radiation source may have enhanced frictional engagement.

In some embodiments, an external attachment mechanism may be utilized. For external attachment mechanism 110′, the circumferential dimension of the attachment device 110′ is necessarily larger than the circumferential dimension of the emitter tube 90, even though the circumferential dimension of the collimator 100 itself may or may not be larger. The device may include a central frame, which may or may not be part of the collimating structure and may be radiation blocking or absorbing, and extension portions that facilitate the actual attachment. These portions may or may not be radiation absorbing or blocking.

FIGS. 4, 4A, 4B and 4C illustrate an example of an external clamping mechanism 110′ for attaching a collimator to an x-ray tube 90. The external clamping mechanism 110′ may generally include an interface 114a′ for attaching to the collimator (not shown), and may employ a plurality of external arms 112′ which may be mounted to a central frame 114′, such as in sliding recesses 114b′. The external arms 112′ may then slide along direction C to clamp down onto the outside of an x-ray tube 90, as illustrated in FIG. 4C. The interface between the external arms 112′ and the sliding recesses 114b′ may include, for example, position retention features, such as ratcheting interfaces. The external arms 112′ may also be translated in the sliding recesses 114b′ using handles, such as the handles 112a′ shown in FIG. 4C.

In a further embodiment, the attachment mechanism may include a central frame having a plurality of concentric cylindrical tubes telescopically connected and biased. The telescopically arranged portions may extend and contract lengthwise to accommodate various sizes of the emitter tube and may translate within each other in a manner similar to an expanding telescopic antenna, and thus the portions may be drawn out until an appropriately sized portion is exposed, as illustrated with the cross-sectional view of the telescoping sections 700 in FIG. 4H. Once the proper size portion is exposed, the device is fixed with a fixing feature, such as a latch, clip or screw, to keep the telescopic portions from collapsing. The outside of the telescopic portion may be used to frictionally engage the inside of an emitter tube or the inside of the telescopic portion may be used to frictionally engage the outside of the emitter tube.

Likewise, the external attachment mechanism may also be formed of similar materials mentioned above for internal attachment mechanism or for the collimator structure.

As mentioned above, the collimator structure 100-1 may in general include a collimator 100 having a main frame 102 with a centrally located aperture 101 through which the radiation may travel, an aperture variation mechanism attached to the main frame 102, and connection portions 206 for connecting the collimator 100 with the receptor holding device 300, as illustrated in FIGS. 1, 1A and 2L. As noted before, an aiming ring 200 may or may not be present. If present, the aiming ring 200 may be interposed between the collimator 100 and the receptor holding device 300, as also illustrated in FIGS. 1 and 1A. The aiming ring may also be fabricated form any of the materials mentioned above for the collimator structure.

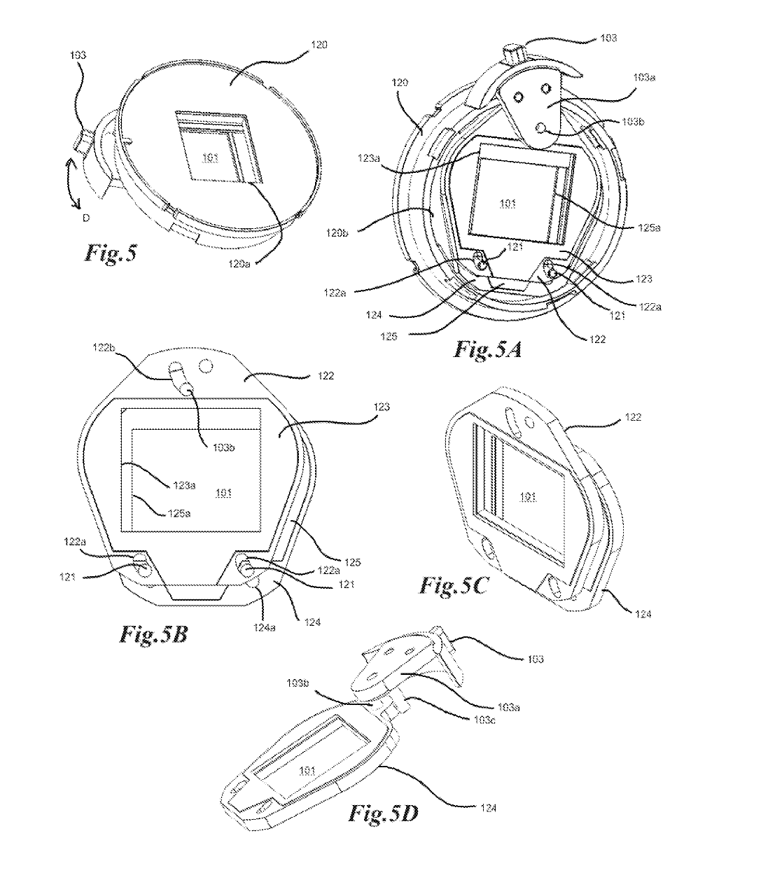

FIG. 5 illustrates an example of an adjustable aperture mechanism which may generally include a primary blocking plate 120 with an opening 120a, where the primary blocking plate 120 may block, attenuate, absorb and/or otherwise substantially prevent passage of radiation therethrough except at the opening 120a. The size and shape of the opening 120a may generally define the maximum dimensions of an aperture 101 for a collimator 100, and may thus define the maximum extent of radiation passing through the collimator 100. The opening 120a may be any appropriate shape and size, and, for example, may be tailored to match the receptor 400 being utilized with the collimator 100. For example, the shape, size and/or operating distance of the radiation receiving element of the receptor 400 may be used to define the shape and size of the opening 120a. This may be desirable as the radiation passing through the opening 120a may substantially incident upon the radiation receiving element of the receptor 400 and not upon other parts and/or as excess radiation that may incident upon other undesired locations or parts of a patient.

As illustrated in FIG. 5, the opening 120a may generally have a rectangular shape as most radiation receiving elements in use are rectangular, such as x-ray film and digital sensors 400, however, other shapes may be employed depending on the receiver being utilized. Also, the overall size of the opening 120a may be tailored, for example, based on the measured and/or projected amount of spread of the radiation beam after passing through the collimator and the operating distance between the aperture 101 and the receptor 400.

In exemplary embodiments, an aperture control, such as the aperture control handle 103 as illustrated in FIGS. 1-2 and 5, may be utilized to adjust the aperture 101. The aperture control handle 103 may generally be used to adjust the size and/or shape of the aperture 101 of the collimator 100. In some embodiments, the aperture control handle 103 may actuate an aperture mechanism, such as a multiple plate aperture mechanism as illustrated in FIG. 5A. In general, multiple plates with openings may be utilized to vary the size and/or shape of an aperture by, for example, varying the degree and orientation of the overlap of the openings. Further in general, the plates may be radiation blocking, attenuating, absorbing, and/or otherwise capable of substantially preventing radiation from passing through them, and may for example, include a main plate body and a radiation blocking layer, insert, cover and/or other separate part for radiation blocking The number of plates utilized may generally be determined by the needed level of adjustability and/or other desired characteristics. For example, 3 plates may be desirable as they may be utilized to adjust the size and shape of an aperture while keeping the position of the aperture centered in a given space.

FIG. 5A illustrates a 3 plate aperture mechanism which includes a primary blocking plate 120 and blocking plates 122, 124 stacked therewith. The plates 120, 122, 124 may further include separate radiation blocking parts, such as the radiation blocking layers 123, 125. For example, for the plates whose main function is radiation blocking or attenuation, they may be constructed of any of the material mentioned above for the collimator structure, for example, metal, metallic material, and/or polymeric material doped with radiation blocking, or absorbing material, such as metal or metal oxides. Examples of suitable metals or metallic materials may include lead, tin or similar. Examples of dopants for polymeric materials may include lead and Bismuth oxides.

The separate layers 123, 125 may be desirable as the radiation blocking portion of the plates need not be the same size as the plates 122, 124 as a whole since, for example, the primary blocking plate 120 may substantially block radiation and the extra blocking from the plates 123, 125 need only be present in areas where aperture 101 is present. The plates 122, 124 may further include openings 123a, 125a, respectively, which may be utilized to overlap with the opening 120a to vary the size and/or shape of the aperture 101. For example, the aperture control handle 103, which may be coupled to a rocker 103a, may be coupled to plates 122, 124 via pins 103c, 103b, respectively, as illustrated in FIGS. 5A, 5B, 5C and 5D. The plates 122, 124 may also include arcuate slots, such as slots 122a, 124a, 122b. Pins 103b, 121 may then guide the movement of the plates 122, 124 with respect to the primary blocking plate 120 with the slots 122a, 124a, 122b and actuation by the aperture control handle 103 in direction D. The plates 122, 124 may further be guided by their shape and interaction with the edges of a depression 120b in primary blocking plate 120. The movement of the plates 122, 124 with respect to primary blocking plate 120 may then vary the aperture 101, with one extent of the aperture control handle 103 generally being a maximum aperture and the other a minimum aperture. For other parts of the plates whose main function is not radiation attenuation or blocking, other materials mentioned above may be used.

The connection between the receptor holding device 300 and the collimator 100 may be effected in various manners. For example, the connection may be effected with complementary features at the connection portion of the elongated arm of the receptor holding device 300 and the outside of the collimator 100, such as illustrated with the mounting fins 104′ on the housing 102′ of collimator 100′ in FIG. 6C. For example, as illustrated, a plurality of complementary slots 104a′ may be utilized to mate to a complementary feature on the receptor holding device 300 such that it may be coupled to the collimator 100′. The shapes of the slots 104a′ and complementary features 104 shown are for illustrative purposes and are not meant to limit their shapes and configurations. For example, the slots 104a′ and complementary features 104 may be of any shapes, as long as they complement each other to facilitate the connection. In addition, the slots and complementary features may also vary in sizes, the size being limited by the size of the overall structure of the device, the obtrusiveness and the aesthetics. The large the size, the more area the contact and the better the connection.

The connection may also be between the receptor holder 300 to a separate aiming ring, if present, which may then also connect to the collimator, as illustrated with a separable aiming ring 102″ that may attach to collimator 100″ in FIG. 6D, such as by snapping on, and/or otherwise being attached or coupled, for example, any slot and complementary feature, protrusion and complementary slot, or similar. This may be desirable if one wish the receptor holder and aiming ring to be utilized without directly coupling to a collimator, such as, for example, when utilizing unusual alignments or angles, or with other collimators and/or no collimator at all. Also, a separate aiming ring which couples to both the receptor holder and to the collimator may be desirable, as distance adjustments can be made with the aiming ring attached to the receptor holder prior to attaching to the collimator, which is stationary on the radiation source and more difficult to move. The aiming ring itself may also include magnetic elements which may, for example, be used to couple magnetically directly to an appropriate metal x-ray tube.

In some exemplary embodiments, a separate aiming ring 200 may be utilized that may include self-guiding and reversible secure connections to both the collimator 100 or 100″ and to the receptor holder 300, as illustrated in FIGS. 1, 1A, 6, 6A, and 6E. As illustrated in FIG. 1, for example, the aiming ring 200 may generally include a ring portion 202 through which there is an opening 201 for radiation from the collimator 100 or directly from the radiation source 90 to pass. The ring portion 202 may further be mounted on an extension 203 which may generally space the ring portion 202 a certain distance away from the front face of the collimator 100 when it is mounted. The aiming ring 200 may further include a collimator interface 204 and a receptor holder interface 206, for example, for mating to the collimator 100 at aiming ring mount 104 and for mating to the receptor holder 300 with attachment rail 306, respectively. The receptor holder interface 206 may, as illustrated, generally include a mounting slot 206a through which the attachment rail 306 may slide to attach and position the receptor holder 300. The collimator 100 may further include multiple aiming ring mounts 104 for attaching the aiming ring at different positions, as illustrated in FIG. 2D with aiming ring mounts 104a and 104b, which may, for example, be used to attach the aiming ring for vertical or horizontal positions. Again, the rail 306 and slot 206a may also take on other shapes and configurations.

In one embodiment, the collimator interface 204 and the aiming ring mount 104 may generally include a complementary interface, such as the nesting block 204a with sloped sides 204b on collimator interface 204 which may nest into a matching depression of aiming ring mount 104, as illustrated in FIGS. 1, 1A and 6E. The complementary interfaces may also include magnetic elements, which may be exposed on the surfaces of the interfaces, or embedded below the surfaces of the interfaces. Magnetic elements may include, for example, permanent magnets, electromagnets, rare earth element magnets, magnetically responsive materials, such as ferromagnetic metals or compounds, magnetizable materials, and/or any other appropriate magnetic material or combinations thereof. For example, some components may include a magnet and the mating complementary component may include a metallic insert or piece which may be attracted to the magnet. This may be desirable as the polarities of multiple magnets may cause unhelpful interference with each other. Also, a magnet on one complementary component may attract a metallic insert or piece on another component while still allowing a greater degree of movement, whereas multiple magnets may generally force themselves to align with each other in a particular position or alignment of maximized magnetic forces, which may make sliding components for different positions difficult. The complementary parts may be nesting such that any action of the magnetic forces of the magnetic elements may generally result in one part nesting into the other in a predicted fashion. This may be desirable as it may serve to minimize or eliminate chances of user error. The nesting parts may also be designed such that only one nesting configuration is possible, which may generally be the proper configuration. The magnetic elements may then generally provide substantially all of the needed force to keep the mated components together and in place while the complementary parts may generally keep the mated components in a desired alignment and/or configuration, and may thus act in concert to create secure and properly aligned connections.

In some embodiments, the collimator interface 204 and the aiming ring mount 104 may generally include a stepped complementary interface, such as the stepped depression 102a around the aiming ring mount 104 as illustrated with the collimator 100″, as shown in FIG. 1D, which is substantially the same as collimator 100, (except that the complementary interfaces for the aiming ring or connecting body are larger and stepped), and the aiming ring 200″ of FIG. 1D with a ring portion 202 mounted on an extension 203 which may generally space the ring portion 202 a certain distance away from the front face of the collimator 100″ when it is mounted. The aiming ring 200″ may further include an enlarged section 210 which may interface and nest into the stepped depression 102a, which may, for example, provide a more secure connection and the larger interface of a particular shape may also aid in properly aligning and attaching the aiming ring 200″ to the collimator 100″ by, for example providing increased visual and physical cues for proper fitting of the components. The enlarged section 210 may also generally fill in the space of the main body 102 to provide a substantially continuous surface when the aiming ring 200″ is attached to the collimator 100″. The aiming ring may also include handles or other handling features 208, to, for example, improve handling of the aiming ring and attachment/removal from the collimator. FIGS. 1B, 1C, 1D, 6F and 6G illustrate an embodiment of an aiming ring 200″ which includes handles 208. For example, the handles 208 may include grip portion which may be roughened or coated for easier grapping by the fingers of the operator. In general, the grip portion may include small bumps distributed in the surface of the handles 208, include a depression in the grip portion, or include a high frictional coating over in the surface of the handles 208. The high frictionally coating may be used for the bumps and the depression as well.

Additional views of the collimator 100″ are shown in FIGS. 2E-2K and additional views of the aiming ring 200″ are shown in FIGS. 6I and 6I-1 to 6I-7.

In some alternative embodiments, the receptor holder 300 and collimator 100 may also mate to a connecting body 200′ rather than a full aiming ring 200, which may not include a ring portion, as illustrated in FIG. 6B. This may be desirable as the connecting body 200′ still provides the ability to easily attach/detach the receptor holder 300 to/from the collimator 100 without the extra ring portion. The ring portion 202 may also be a removable attachment to the connecting body 200′ and may be utilized whenever a ring is desired or needed by the user. The connecting body 200′ may also connect to the collimator 100 at the multiple aiming ring mounts 104a, 104b as illustrated in FIG. 2D, for mounting the receptor holder 300 at either a horizontal or vertical position on the collimator 100. The aiming ring mounts 104a, 104b may, for example, be substantially 90 degrees separated on the collimator 100 such that when an aiming ring 200 or connecting body 200′ is used to mount a receptor holder 300, the receptor holder 300 may be oriented either vertically or horizontally relative to the collimator 100.

In some other alternative embodiments, the receptor holder 300 and collimator 100 may also mate to a connecting body 200′″ rather than a full aiming ring 200″, which may not include a ring portion, as illustrated in FIGS. 1D, 6F and 6G. This may be desirable as the connecting body 200′″ still provides the ability to easily attach/detach the receptor holder 300 to/from the collimator 100″ without the extra ring portion. The ring portion 202 may also be a removable attachment to the connecting body 200′″ and may be utilized whenever a ring is desired or needed by the user. The connecting body 200′″ may also generally include handles 208 as in the aiming ring 200″ and/or the enlarged section 210 for mating to the stepped depression 102a of the collimator 100″. The connecting body 200′″ may also connect to the collimator 100″ at one of the multiple aiming ring mounts 104a, 104b with the enlarged section 210 mating to one of the stepped depressions 102a, 102b, as illustrated in FIG. 1E, for mounting the receptor holder 300 at either a horizontal or vertical position on the collimator 100″. The aiming ring mounts 104a, 104b may, for example, be substantially 90 degrees separated on the collimator 100″ such that when an aiming ring 200″ or connecting body 200′″ is used to mount a receptor holder 300, the receptor holder 300 may be oriented either vertically or horizontally relative to the collimator 100″.

FIGS. 6H and 6H-1 illustrate how the aiming ring or connecting body may be connected to the collimator at either a vertical or horizontal position, as discussed above, with an example shown with collimator 100″ mounting an aiming ring 200″ at either the horizontal position with aiming ring mount 104a in FIG. 6H or at the vertical position with aiming ring mount 104b in FIG. 6H-1. The different positions may also be used with the collimator 100, the aiming ring 200, the connecting bodies 200′ and 200′″. It may be appreciated that these arrangements may produce a variety of different orientations of the receptor depending on which aiming ring mount and which receptor holding device is used.

The aiming ring 200 or 200″, as noted above, may be a separate structure for attaching to the collimator 100 or 100″ and the receptor holding device 300; or the aiming ring 200 or 200″ may be an extension integrally formed on and/or formed as a detachable piece for the aiming ring portion of the elongated arm of a receptor holder 300. Also, the aiming ring 200, whether separate or integral to the collimator 100 or 100″, may be fabricated from the same or similar materials used in the collimator 100 or 100″ mentioned above.

Likewise, the connecting body 200′ or 200′″ may also be a separate structure for attaching to the collimator 100 or 100″ and the receptor holding device 300; or it may be an extension integrally formed on and/or formed as a detachable piece for the connecting body 200′ or 200′″ portion of the elongated arm of a receptor holder 300.

Sensors are getting more and more sensitive to radiation so that a smaller dosage or a weak radiation source may be used. For use with the traditional source and a highly sensitive sensor, a filter such as an attenuating filter may be used. In some other embodiments, the aiming ring may be utilized to mount a filter or other object in the line of the X-rays emitted from the aperture 101. For example, an attenuating filter may be placed in the ring 202 for reducing the overall amount of X-rays delivered to the patient and the receptor. Polarizing gratings may also be utilized to, for example, aid in directing X-rays toward a particular target and to reduce scattered X-rays emanating from the X-ray emitter 90.

For example, as mentioned above, useful sensors may include those using scintillation technology may enable high resolution image capture with increased contrast ratio, resulting in more diagnostic detail in the radiograph. For another example, CMOS image detector chip with 18 bit image capture may also result in higher resolution and reduced pixilation when zooming in on small details.

Though the higher sensitivity sensors are useful, the present invention is not limited to those, but is also useful for X-ray films, lower sensitivity sensors and so on.

Useful sensors may also include those commercially available, or those with ergonomically designed body with rounded corners and edges without clipping the corners of the active area, resulting in a more comfortable sensor without the loss of anatomical coverage.

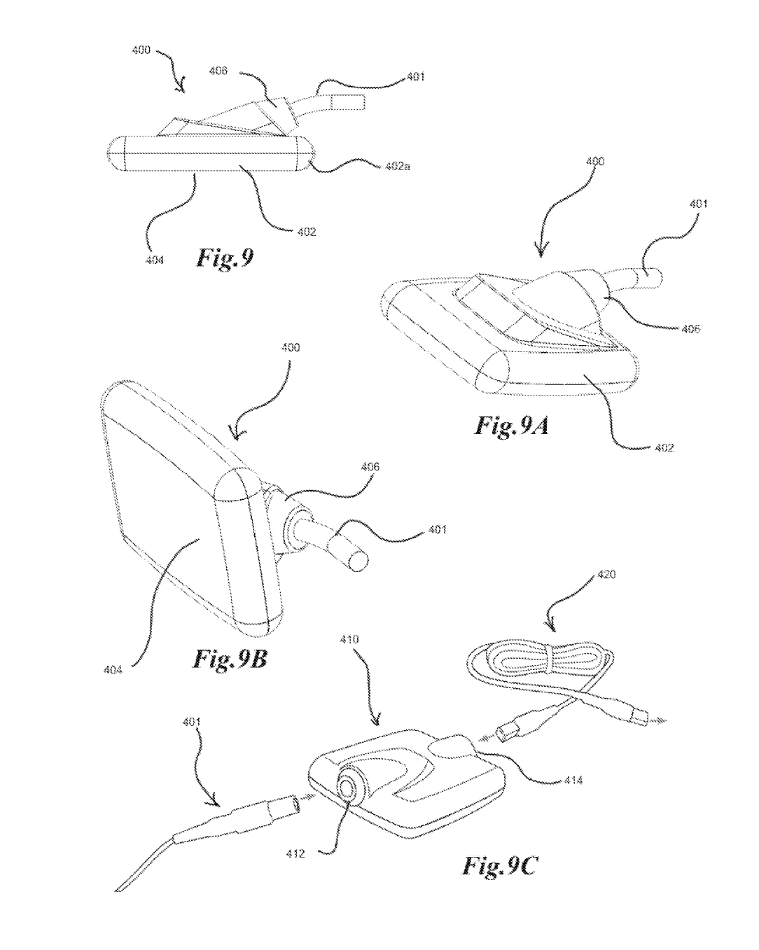

FIGS. 9, 9A, and 9B illustrate an embodiment of a receptor 400 which may be an x-ray sensor. The receptor 400 may generally include a body 402 which may interface with a holding portion of a receptor holding device, such as any of those described above, and which may further include internally an x-ray sensitive sensor, which may generally have an active area from which it may receive x-ray radiation, such as the active area 404, which may generally face the x-ray emitter 90 during use, as shown in FIG. 1. The body 402 may generally have rounded edges and/or corners, such as corners 402a as illustrated, to, for example, provide a more comfortable handling to a user and/or patient. The receptor 400 may further include a cable 401 for powering and/or transmitting data to/from the x-ray sensitive sensor. The cable 401 may generally emerge from a mounting on the back side of the receptor 400, such as from mounting 406, which may, for example, emerge at an angle to aid in reducing fatigue or usage damage to the cable, such as, for example, by emerging at, for example, about 20 to about 90 degrees, more for example, about 25 to about 60 degrees, even more for example, about 30 to 50 degrees, still more for example, about 30 degrees. The receptor 400 may interface to a computer or other device via the cable 401. In some embodiments, the cable 401 may plug into an interface, such as a USB or other computer connector. The cable 401 may also plug into an interface box 410 at a port 412, as illustrated in FIG. 9C. The interface box 410 may serve as an adapter for a different type of cable to output to a computer, such as with the USB cable 420 plugging into port 414, as illustrated.

For taking dental X-rays, for example, bitewing radiographs of a patient's teeth, the collimator structure 100-1 having an opening 101 therein may first be coupled to the radiation source, such as an X-ray emitter 90. A receptor holding device 300 with a holder having a receptor 400 for exposing X-ray from the X-ray emitter may then inserted into a patient's oral cavity prior to coupling it to the collimator 100.

The commonly prescribed dental radiograph such as the “bitewing”, is an image that is acquired of the crowns of the teeth biting together and their surrounding socket bone. Generally, the film or sensor 400 mounted in a receptor holding device 300 when inserted into a patient's oral cavity, is held in place when the patient bites down on the bite portion with the target teeth and holds the film or digital sensor 400 in position next to the target.

As mentioned above, capturing intraoral images of a target, such as a patient's teeth, on a receptor 400, for example, film or electronic sensor, such as a digital sensor, involves positioning a film or sensor 400 within a patient's oral cavity, for example, next to the inner surface of the teeth or bone being studied. The film or sensor 400 may then be exposed to radiation, for example, an X-ray beam, generated outside the oral cavity and passing through the target.

Both film and electronic sensors are widely used. The electronic sensor technology is a more recent development than the film and may have added advantages over the older film technology. With film, almost everything needed for imaging is packed into the film packet. It is compact and simple, images are not instantaneously available for viewing after exposure. The film is developed offline first prior to viewing. With electronic sensors, the sensors are connected to a computer system, either via wired or wireless connections. With this connection, the images of the teeth may be almost instantaneously generated by the computer without the film developing process. Also, the operator may quickly determine in real time if the image is the one required for proper diagnosis of the patient or if further imaging is required. In addition, because the images are generated electronically, they can be stored electronically in a computer database. In addition, the electronic sensor may be generally made to be more sensitive to X-rays than film, allowing the dosage of x-rays to the patient to be lowered. Examples of film-less dental radiography systems, generally referred to as digital dental x-ray devices, may include those described in U.S. Pat. No. 4,160,997, U.S. Pat. No. 5,434,418, and U.S. Published Patent Application No. 2003/0156681, the contents of all of which are hereby incorporated by reference in their entirety. As mentioned above, communication between the sensor and the computer may be wired or wireless (as disclosed in U.S. Published Patent Application No. 2004/0005032, the content of which is hereby incorporated by reference in its entirety). Nevertheless, the present invention is related to both film and film-less radiography.

According to the present invention, the system, as shown and illustrated in FIG. 1A, 2L, 7 and 7A, may include a receptor holding device 300 having a receptor holder 304 for retaining a receptor 400, for example, an X-ray film or electronic sensor, such as a digital sensor, at one end of an elongated arm 302, a connection portion, such as an attachment rail 306 to a collimator 100 at another end, and a bite portion 302a, which may be a portion of the elongated arm 302, disposed between the ends. The bite portion 302a may be positioned generally more towards the holder 304 end than the attachment portion 306 on the elongated arm 302. The elongated arm 302 from the bite portion 302a further extends to couple to the receptor holder 304 or has a receptor holder portion 304 already integrally formed on it, such that an unobstructed path is defined between the opening or aperture 101 of the collimator 100 and the receptor 400 in the receptor holder 304 while the patient's teeth engage the biting portion 302a of the elongated arm 302.

In one embodiment, the receptor holding device 300 includes an elongated arm 302, a receptor holder portion 304 towards one end of the elongated arm, a bite portion 302a close to the holder on the elongated arm 302 and a connection portion, such as an attachment rail 306, towards the other end, for example, as shown in FIGS. 1, 1A, 2L, 7 and 7A. The receptor holding device 300 may also include features for holding and/or retaining a cable or wire, such as the cable guides 308, for use in situations such as when the receptor 400 includes a cable 401.

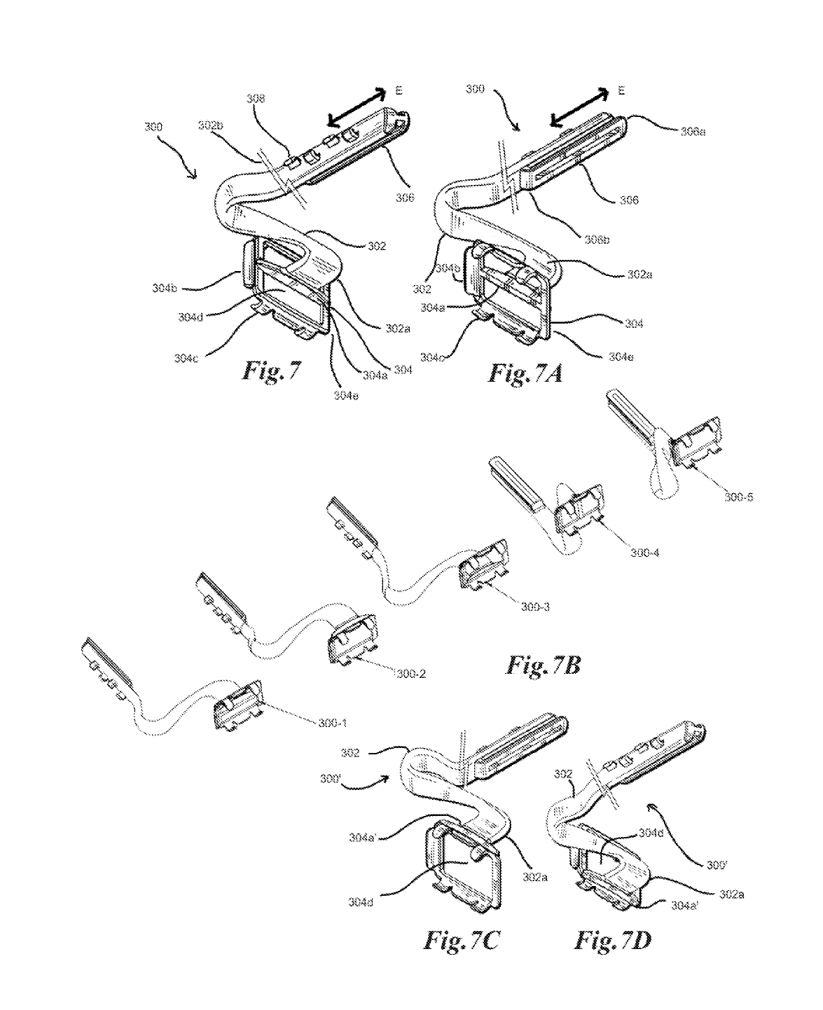

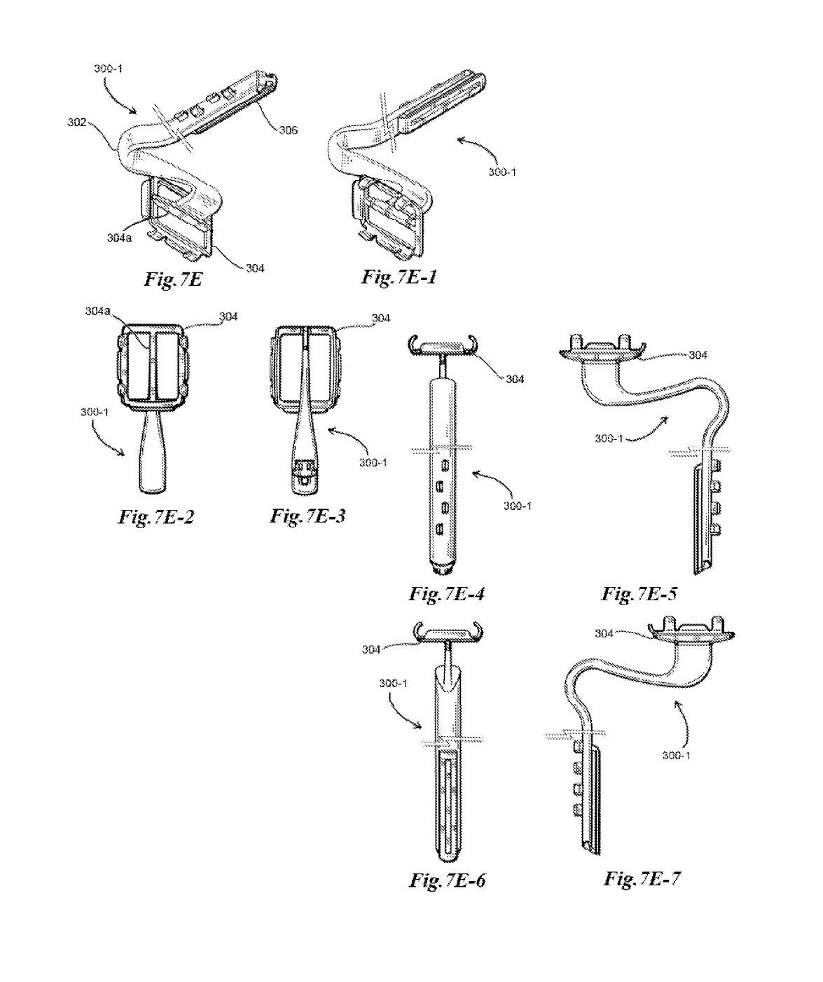

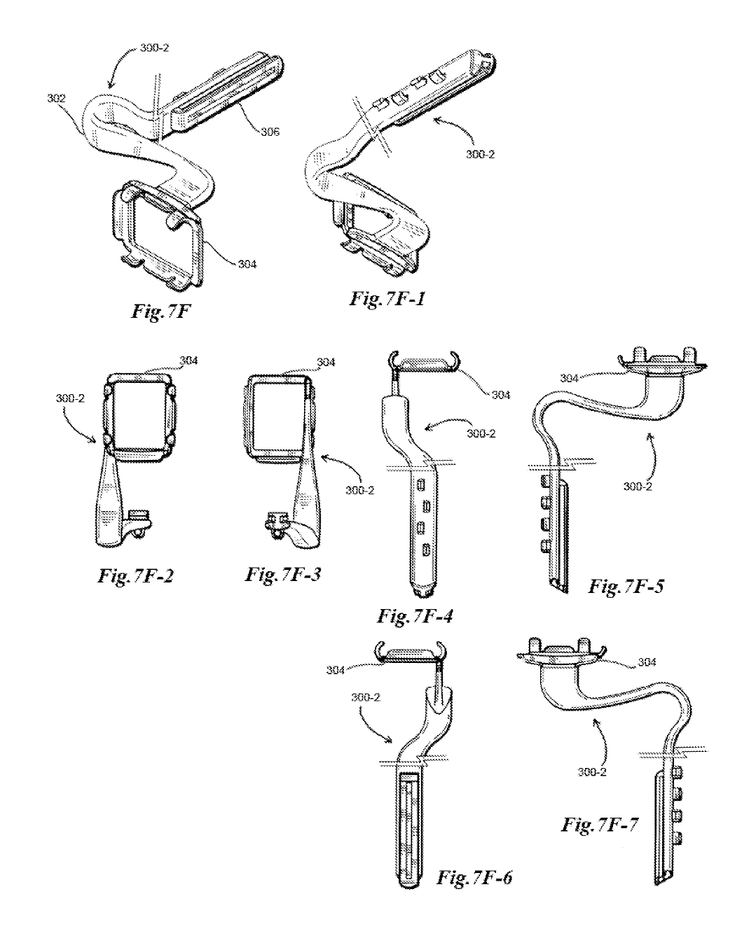

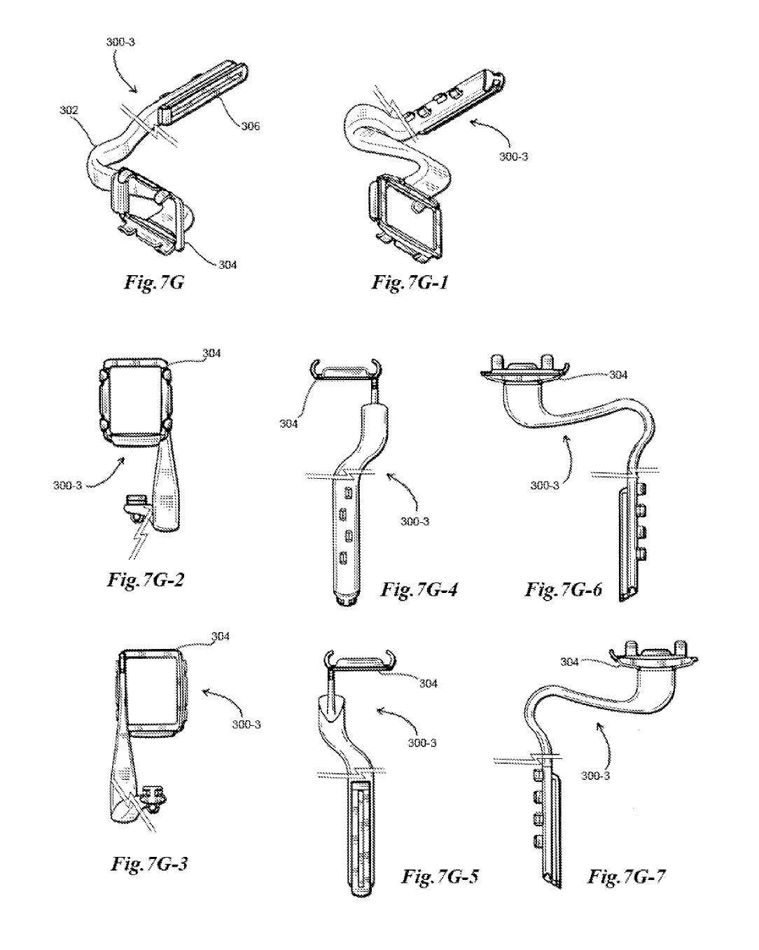

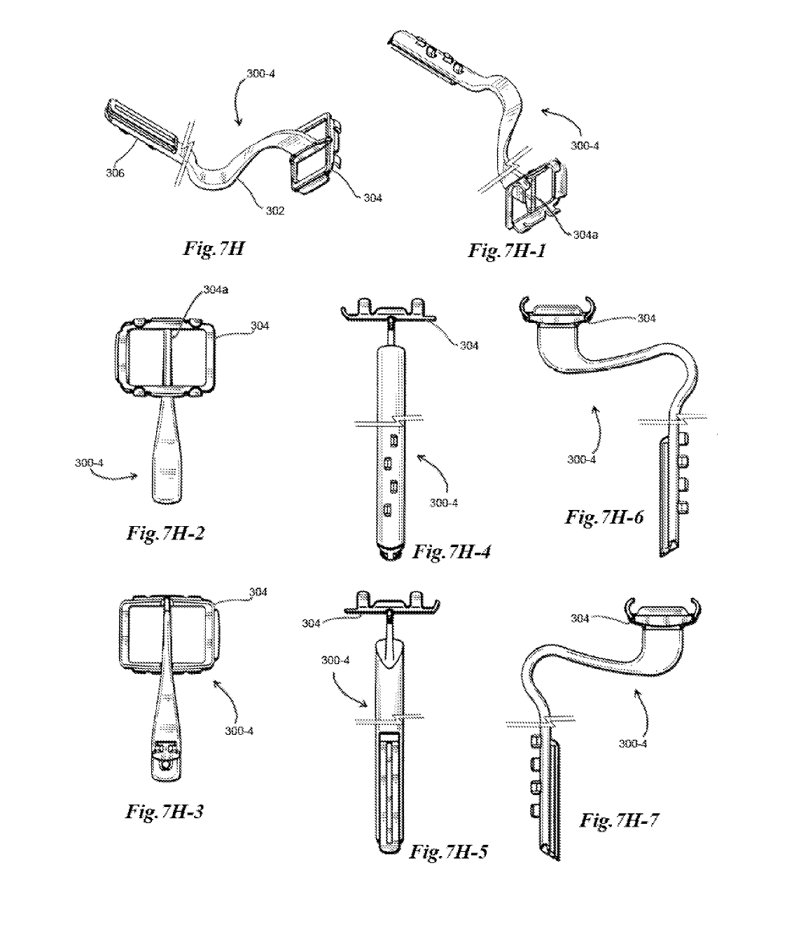

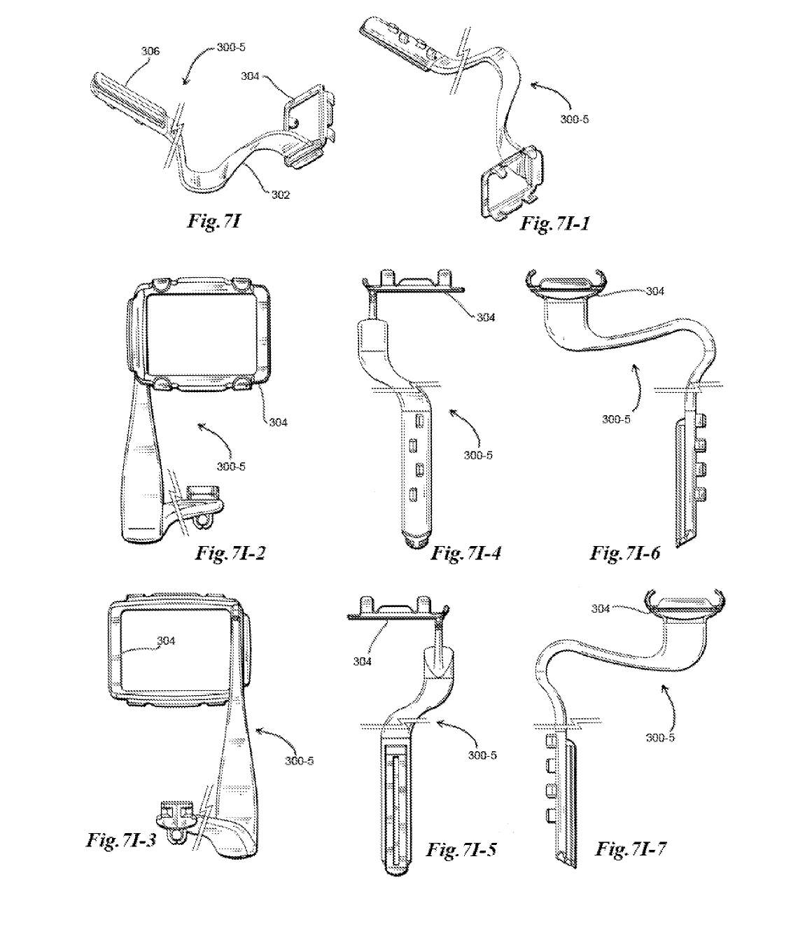

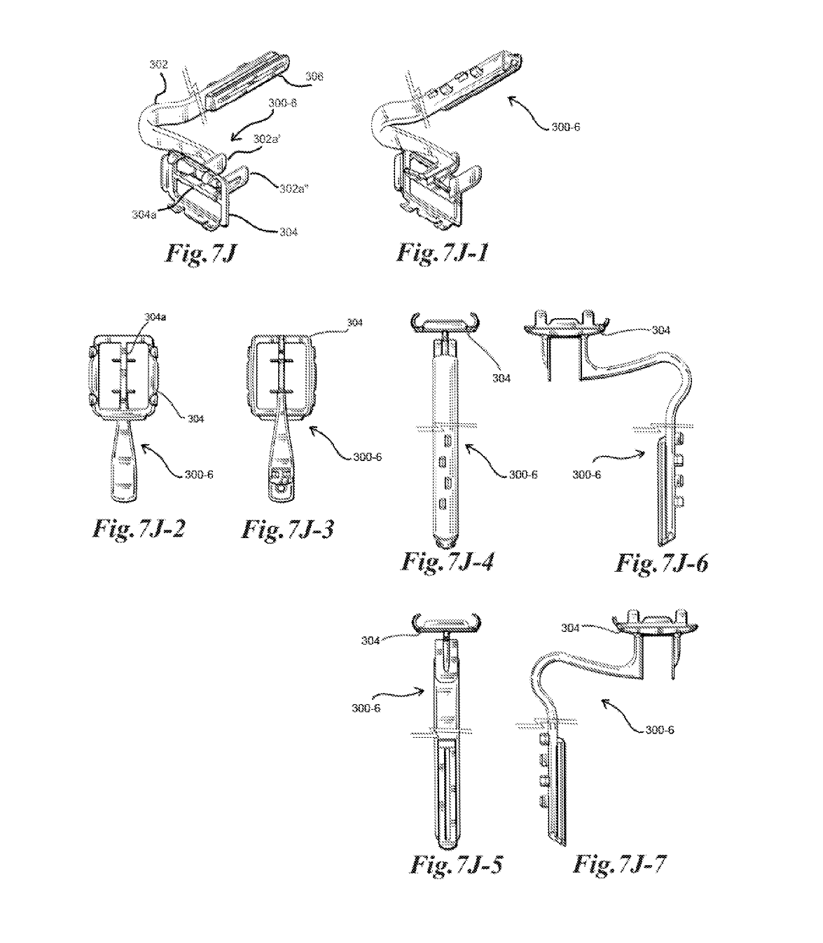

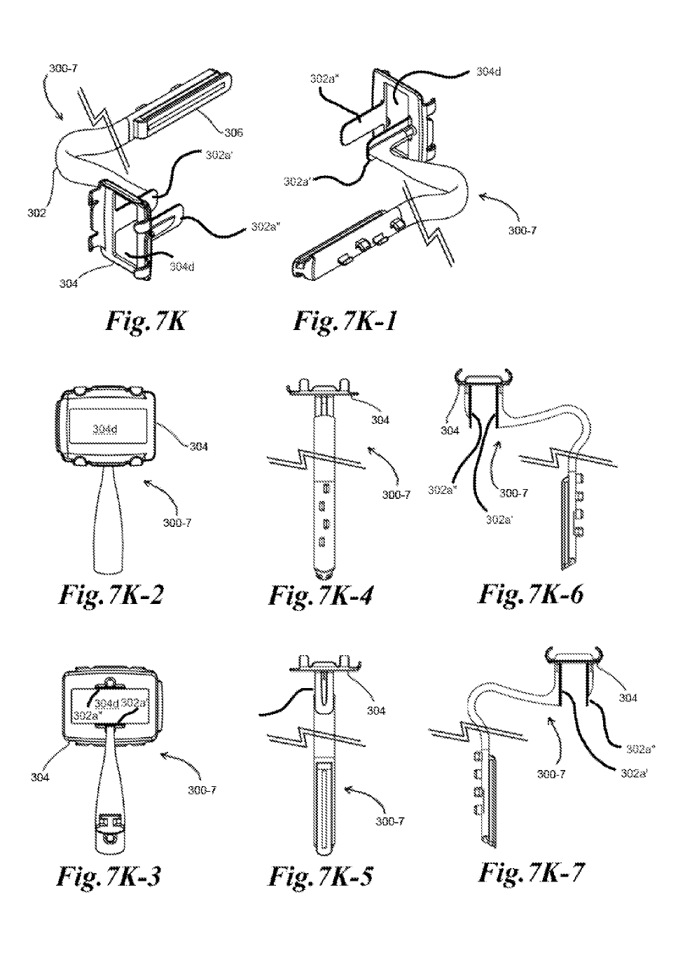

The connection portion, such as the attachment rail 306, may generally be shaped to couple to either the collimator 100 directly and/or at receptor holder interface 206 of an aiming ring 200 or a connecting body 200′. For example, the shape of the attachment rail 306 may generally be complementary and/or match the shape of slots 206a or 104a′, such that the rail 306 may be inserted into the slot for coupling with insertion end 306a. As noted above, the shape and configuration of the complementary attachment features may be of any shape and size and not limited to what are illustrated here, as long as they operate to facilitate a secure connection during use and easy detachment after use. The attachment rail 306 may generally slide back and forth in the slot, which may be utilized to vary the distance of the receptor 400 from the radiation source 90 during use, such as illustrated with sliding direction E in FIGS. 1A, 7 and 7A. The attachment rail 306 may also generally include a stop 306b which may define one extreme of the sliding along direction E.

In some exemplary embodiments, the receptor holder 300 may include self-guiding and reversible secure connections to the collimator 100 and/or to the aiming ring 200, as illustrated in FIGS. 1, 1A, 2L, 6, 6A, 7 and 7A, and as discussed above with regard to the aiming ring 200. The receptor holder interface 206 may, as illustrated, generally include a mounting slot 206a through which the attachment rail 306 may slide to attach and position the receptor holder 300. Self guiding systems may generally include magnetic materials or elements, as discussed above.

In one embodiment, the complementary interfaces, such as slot 206a and attachment rail 306, may also include magnetic elements, which may be exposed on the surfaces of the interfaces, or embedded below the surfaces of the interfaces. The complementary parts may be nesting such that any action of the magnetic forces of the magnetic elements may generally result in one part nesting into the other in a predicted fashion. In an exemplary embodiment, the initial insertion of the attachment rail 306 into slot 206a may generally cause the magnetic elements of both complementary parts to come into proximity, and the attractive force of the magnetic elements may then, for example, draw the attachment rail 306 fully into the slot 206a. This may be desirable as it may serve to minimize or eliminate chances of user error and may also enhance the ease of inserting the attachment rail 306. The magnetic elements may then generally provide substantially all of the needed force to keep the mated components together and in place while the complementary parts may generally keep the mated components in a desired alignment and/or configuration.

As can be seen from FIGS. 7, 7A and 7B, each variation of the receptor holding device 300 may include an elongated arm 302 which may take on various configurations for taking images of different parts of the oral cavity, such as with the receptor holders 300-1, 300-2, 300-3, 300-4 and 300-5 as illustrated in FIG. 7B and with further views illustrated in FIGS. 7E, 7F, 7G, 7H, 7I (for anterior imaging, for example) and 7J (for endodontic imaging, for example), respectively, while generally retaining the same features, portions and components besides the shape of the elongated arm 302 and the manner of the elongated arm interfacing with the receptor holding portion 304. The S-like curve of the arm may vary, for example, from a receptor holding device for taking an anterior image of the oral cavity to a receptor device for taking a posterior image of the oral cavity. For example, receptor holding device 300-1 and receptor holding device 300-4 may typically be a horizontal middle holder and a vertical middle holder, respectively, where the elongated arm 302 interfaces with the receptor holder portion 304 at a crossbar 304a which may be between the upper and lower teeth when used, as the patient bites down on the bite portion 302a. The crossbar 304a and the bite portion 302a may also be generally positioned and shaped such that a minimal amount of the components may lie in front of the patient's teeth during use, such that they may cast minimal shadows on the receptor 400 for radiation from the radiation source 90. For further example, receptor holder 300-2, 300-3, and 300-5 may typically be horizontal left, horizontal right, and anterior holders, respectively, and may generally have elongated arms 302 which interface with the receptor holding portions 304 at the edges without the use of a crossbar, such that the elongated arms 302 and the bite portions 302a thereof do not lie in front of the radiation receiving portion of the receptor 400 during use. The various S-configurations may also generally vary in overall length of at least a portion of the elongated arm 302, such as at varying length zone 302b. This may be desirable, for example, to accommodate different size mouths of patients.

The variations in the configurations of S-like curve portions of the receptor holding arms facilitate in keeping an unobstructed path in the line of sight between the collimator aperture and the receptor for various views and sizes of the image without substantial changes to the rest of the receptor holding device.

In general, the horizontal middle holding device 300-1 may be used to take bitewing radiographs of the posterior teeth of Upper (Maxillary) AND Lower (Mandibular) arches in the same view.

In general, the horizontal left holding device 300-2 may be used to take periapical radiographs of the posterior teeth of the Upper (Maxillary) Left arch and then flipped 180 degrees to take radiographs of the posterior teeth Lower (Mandibular) Right arch only.

In general, the horizontal right holding device 300-3 may be used to take radiographs of the posterior teeth of the Upper (Maxillary) right arch and then flipped 180 degrees to take radiographs of the posterior teeth of the Lower (Mandibular) left arch only.

In general, the vertical middle holding device 300-4 may generally be used in the same manner as the horizontal middle holder 300-1, but in a portrait orientation rather than a landscape orientation, for example.

In general anterior holding device 300-5 may be used to take periapical radiographs of the anterior teeth of the Upper (Maxillary) arch and then flipped 180 degrees to take radiographs of the anterior teeth of the Lower (Mandibular) arch only.

In another embodiment, a receptor holder may be utilized in endodontic procedures. FIGS. 7J and 7J-1 to 7J-7 illustrate an example of a receptor holder as endodontic holding device 300-6. In general, endodontic holding device 300-6 may be used when taking radiographs of a single tooth that is undergoing a root canal procedure. Fins are generally present on the endodontic holding device 300-6, such as fins 302a′ and 302a″ shown attached to crossbar 304a of the receptor holder portion 304. To follow the progress of the endodontic procedure, the dentist may typically need to take radiographs to see the progress of the endodontic file that is being inserted into the canal inside a tooth, so as to make sure the file is going in the correct direction. A certain portion of the endodontic file generally sticks out of the tooth undergoing the procedure, and the fins, such as fins 302a′ and 302a″, may serve as spacers between the two arches of teeth such as, for example, to keep the two arches separated by a given distance, such as the distance between the fins 302a′, 302a″, to keep the file from contacting the opposing teeth. The fins 302a′ and 302a″ may vary in dimension and orientation to, for example, accommodate different size files, different depths of file insertion in the tooth, angle or orientation of the file or tooth, different sized sensors or x-ray film, and/or any other variations in endodontic procedures. A further example of an endodontic receptor holding device is illustrated in FIGS. 7K and 7K-1 to 7K-7 with endodontic holding device 300-7, which illustrates a portrait orientation of the receptor holder portion 304 and the fins 302a′ and 302a″ mounted such that they do not substantially impinge on the active area of a receptor or x-ray film. For example, as illustrated, the fins 302a′ and 302a″ may be mounted to the sides of the receptor holder portion 304, which may be desirable to keep the fins 302a′ and 302a″ out of the opening 304d and thus out of the line of sight from the x-ray source to the active area of a receptor or x-ray film.

The various S-configurations provide needed alignment with the collimator structure, as well as keeping as much of the receptor holders out of the line of radiation from the radiation source 90 to the receptor 400 during use. Each configuration of the receptor holding device 300 may be easily connected to the collimator structure 100-1 without any adjustment prior to imaging, thus, improving the ease and accuracy of the imagining process.

The present invention also provides a device having an unobstructed path between the collimator aperture or opening 101 and the receptor 400. By unobstructed it also means that there is minimal extraneous material, be it material that does not interfere or distort the radiation to any significant extent. Although the receptor holder is generally made of a material having minimal interference with or distortion of radiation, it may nevertheless cause, although minute, artifacts and/or distortions in the imaging process.

In one embodiment, the receptor holder 300 of the present invention includes a picture frame-like structure for retaining the receptor 400, such as the receptor holding portion 304 as illustrated in FIGS. 1, 7 and 7A, without intruding into the active areas of the receptor 400. In general, the active and/or radiation receiving area of the receptor 400 may be smaller than the overall size of the receptor 400. For example, there may generally be a border around the edge of the receptor 400 that may be non-active and thus not used for receiving radiation and/or acquiring images. The retention may be effected with, for example, at least two, more for example, four claw-like features 304c extending from the edges of the main body of the frame-like structure to grasp the receptor about the peripheral, without intruding into the active area. FIGS. 7 and 7A illustrate an example of a receptor holding portion 304 which includes multiple claw-like structures 304c for grasping the receptor 400 when it is inserted at open end 304e. The receptor holding portion 304 may also generally include an opening 304d which may generally match the size, shape and position of the active area of the receptor 400 when it is inserted. The receptor 400 may also generally be inserted and stopped by end retaining structure 304b. The retaining structures and/or the receptor holding portion 304 as a whole may generally be formed to grasp the receptor 400 firmly yet reversibly, such as by friction and/or by complementary formations of the receptor 400, such as depressions or grooves.

The elongated arm 302, either separate or integrally formed with the receptor holding portion 304, may also extend from behind and/or to the side of the receptor 400 so that it does not intrude into the path of radiation. The elongated arm 302 may extend from the lower edge of the holder, the upper edge of the holder or from the mid-section of the holder. For the embodiments with a crossbar 304a across the mid-section of opening 304d, the elongated arm 302 may extend from the crossbar 304a.

In some embodiments, the receptor holding portion 304 may include a horizontal crossbar 304a extending across the mid-section of the picture frame-like structure, such as by bisecting the opening 304d as illustrated in FIGS. 7 and 7A. The receptor 400 may rest with its back portion, i.e. the portion not facing the radiation source 90, against the crossbar 304a when installed so that the crossbar 304a does not intrude into the path of radiation. Also, as discussed above in conjunction with receptor holders 300-1 and 300-4, the crossbar 304a may also lie in the same line as the bite portion 302a and may be utilized with the patient's upper teeth and lower teeth on either side of the crossbar 304a such that it does not substantially obstruct the usable active part of the receptor 400.

In other embodiments, such as illustrated with receptor holder 300′ in FIGS. 7C and 7D, and with receptor holders 300-2, 300-3, 300-5 in FIG. 7B, the elongated arm 302 may interface with the receptor holding portion 304 at an edge, such as edge 304a′, such that the elongated arm 302 and its bite portion 302a may lie wholly or substantially out of line with the opening 304d.

The connection portion of the elongated arm 302 that connects the receptor holding device 300 to the collimator structure 100-1 may include various attachment features. These features may mate with receptacles on the collimator structure 100-1 to form a secure connection during operations and detachment without excessive effort after imaging is completed. Mating of the attachment features of the receptor holding device 300 with the receptacles on the collimator structure 100-1 may be effected in various ways, including electrical, mechanical, optical, fiber optic, magnetic or of other similar connection types that satisfy the secure attachment and easy detachment criteria. Also, the receptacles may be present in multiple places on the collimator structure to accommodate mating with different receptor holding devices, for example, for taking anterior or posterior images, while keeping proper alignment for imaging.

In one embodiment, a magnetic connection may be effected.

In other embodiments, features such as contact sensor receptacles, may be used to form a purely mechanical connection between the image receptor holding device and the collimator 100. In still other embodiments, contact sensor receptacles may be used in addition to other connection features, to ensure that a receptor holding device is attached properly before firing x-ray generator. In yet other embodiments, one or more lights may be used and their illumination may be used to indicate the status of the connection, such as green to indicate a proper connection with collimator 100 has been made and the x-ray generator is ready to fire, and red to indicate the x-ray generator is not ready to fire, for example. In other example, a light may be illuminated to indicate that a proper connection has been made, and the light is not illuminated when a proper connection is not made. In yet more embodiments, an audible sound, for example, a beeping sound, may be emitted to indicate that a proper connection has been made.

To maintain the structure and orientation of the various receptor holding devices, they may be fabricated from materials that are sufficiently rigid without having too much thickness. The materials mentioned above for the fabrication of the collimator structure or attachment mechanism, for example, the arms, may be suitable for making the receptor holding devices.

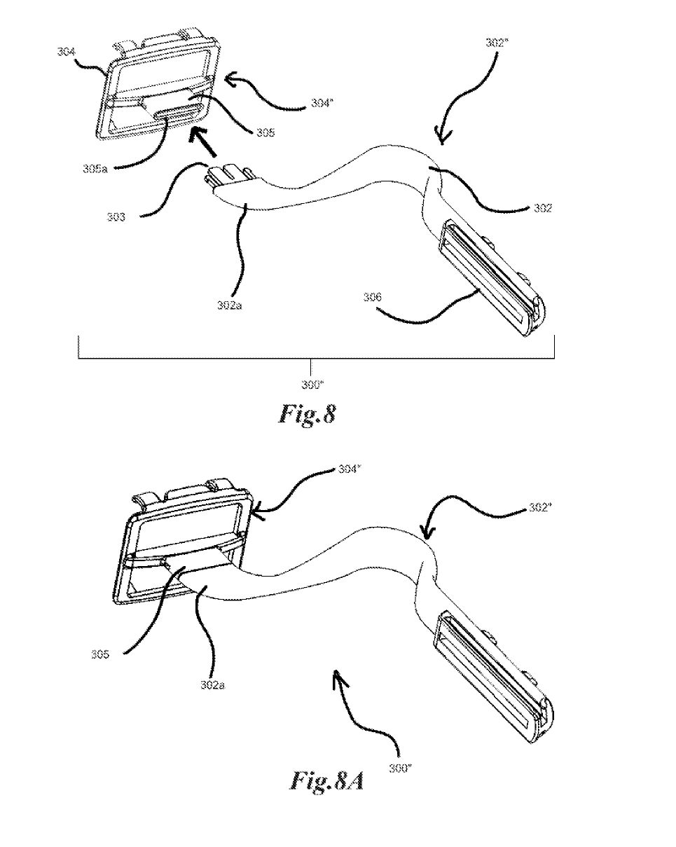

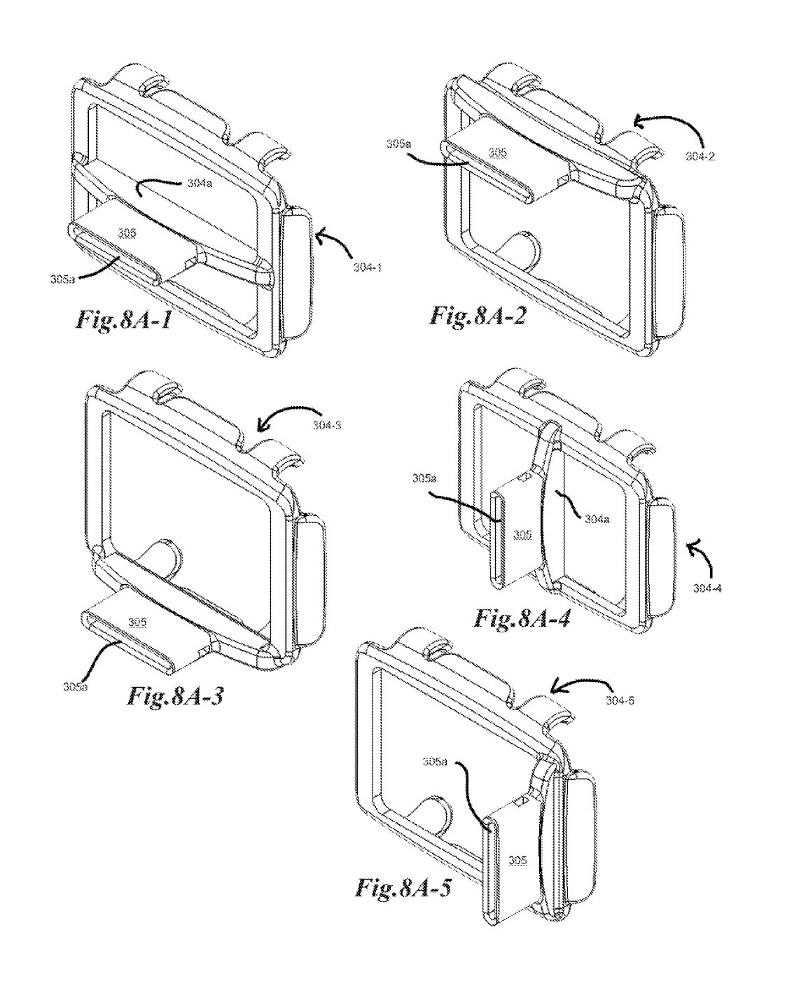

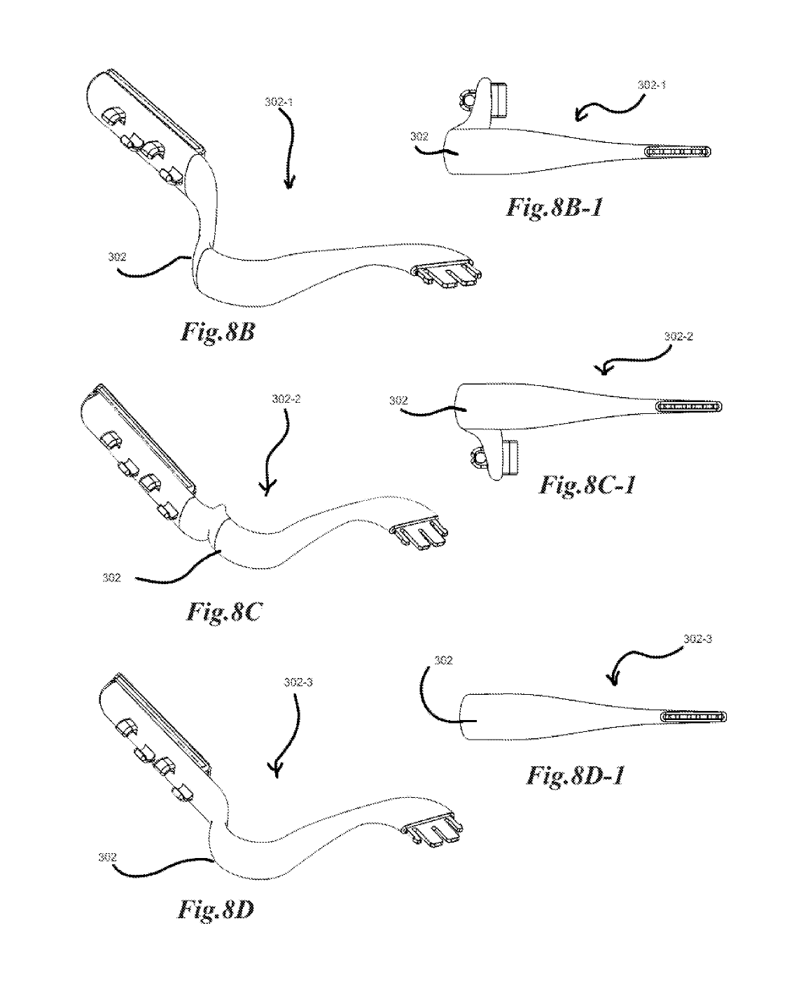

In some embodiments, a receptor holder may include multiple components which may be assembled to form the complete receptor holder. FIGS. 8 and 8A illustrate an example of a receptor holder 300″ which may include an arm portion 302″ and a holder portion 304″, which may be assembled to form a complete receptor holder which may be substantially the same as a unitary piece receptor holder, such as the receptor holder 300 of FIGS. 7 and 7A. For example, FIG. 8 illustrates the arm portion 302″ with a snap-on fitting 303 on the end of the bite portion 302a which may be inserted as shown into a slot 305a of the bite portion 305 of the holder portion 304″, and thus may form the complete receptor holder 300″ in FIG. 8A. In general, any appropriate fitting and/or coupling features or mechanisms may be used to assemble the holder portion 304″ and the arm portion 302″, such as, for example, snap fittings, friction fittings, compressible fittings, screw on fittings, pinned fittings, and/or any other appropriate fitting.

In general, each of the receptor holders described above, including the receptor holders 300-1, 300-2, 300-3, 300-4, 300-5 may be formed as a multiple piece receptor holder such as the receptor holder 300″. FIGS. 8A-1, 8A-2, 8A-3, 8A-4, 8A-5 illustrate the holder portions 304-1, 304-2, 304-3, 304-4, 304-5 which may be used to assemble a receptor holder 300-1, 300-2, 300-3, 300-4, 300-5 as shown in FIG. 7B, respectively. In general, the arm portion of the receptor holder may take on one of a few shapes and at least one arm portion may be useful in forming more than one receptor holder such as 300-1, 300-2, 300-3, 300-4, 300-5. FIGS. 8B and 8B-1 illustrate an arm portion 302-1 with a dropped arm section 302, which may be coupled with holder portion 304-3 to form receptor holder 300-3. FIGS. 8C and 8C-1 illustrate an arm portion 302-2 with a raised arm section 302, which may be coupled with holder portions 304-2 or 304-5 to form receptor holders 300-2 or 300-5, respectively. FIGS. 8D and 8D-1 illustrate an arm portion 302-3 with a no-rise arm section 302, which may be coupled with holder portions 304-1 or 304-4 to form receptor holders 300-1 or 300-4, respectively. Other combinations of the arm portions and holder portions may also be utilized to produce varied receptor holder configurations.

While exemplified embodiments of the invention have been described and illustrated above, it should be understood that these are exemplary of the invention and are not to be considered as limiting. Accordingly, the invention is not to be considered as limited by the foregoing description, but is only limited by the scope of the claims appended hereto.

1. An imagining system for imaging a portion of an oral cavity onto a receptor on a holding device, comprising:
a collimator structure comprising a central frame having an attachment mechanism on one end of said central frame, said central frame having an outside surface and an aperture therein; and
an aperture adjustment mechanism disposed in said central frame for varying a size and/or shape of said aperture;
wherein the outside surface of said central frame comprises a plurality of features, at least one of said plurality of features being adapted for magnetically mating with an attachment portion on said holding device to create an unobstructed line of sight between the aperture and the receptor during imaging.

2. The imaging system of claim 1 wherein one of said features comprises a portion of a connecting body portion positioned at a second end of said central frame for attachment to said holding device.

3. The imaging system of claim 2 wherein said connecting body portion is an integral part of an aiming ring portion or a separable component.

4. The imaging system of claim 3 wherein said connecting body portion comprises a plurality of features, said features being complementary to at least one of the features on the frame and the holding devices for magnetically mating with said frame and said holding device.

5. (canceled)

6. The imaging system of claim 4 wherein at least one of said features comprise a handle for ease of grasping the aiming ring during coupling with said central frame.

7. The imaging system of claim 1, wherein at least one of said features comprise a magnetic material.

8. (canceled)

9. (canceled)

10. The imaging system of claim 1 wherein said attachment mechanism comprises:
at least three window-like formations disposed substantially equal distance from one another thereon said central frame;
at least three arm-like structures, each having a pivotal edge, a non-pivotal edge, and a substantially arc-like exterior surface in between, mounted inside each of said window-like formations along the pivotal edges for pivoting outward from said frame;
at least two plates rotationally coupled to each other for rotation in opposite angular directions for pivoting the arm-like structures with respect to said frame; and
at least one biasing member mounted on said frame for biasing the arms in an outward pivot.

11. (canceled)

12. (canceled)

13. (canceled)

14. (canceled)

15. (canceled)

16. The device of claim 10, wherein said biasing member comprises a spring.

17. The imaging system of claim 1 wherein said aperture adjustment mechanism comprises:
a central frame comprising an aperture control positioned thereon;
at least one fixed plate mounted to said central frame; and
two smaller movable plates stacked on top of said large plate, said two smaller movable plates each having an aperture.

18. (canceled)

19. (canceled)

20. (canceled)

21. A receptor holding device comprising:
an elongated arm having a curved portion;
an attachment portion disposed on said elongated arm towards one end of said elongated arm adapted for attaching to an interface;
a receptor holder portion disposed on said elongated arm towards a second end of said elongated arm;
and
a receptor holder disposed on said receptor holder portion.

22. The receptor holding device of claim 21 wherein said receptor holder comprises a frame-like structure for retaining a receptor having an active area unobstructed by said holder.

23. The receptor holding device of claim 21, wherein said curved portion of said elongated arm comprises different shapes for accommodating different receptors for imaging different parts of an oral cavity.

24. The receptor holding device of claim 21, wherein said attachment portion comprises a magnetic attachment feature for mating with a complementary feature in the interface.

25. The receptor holding device of claim 21, wherein said attachment portion is adapted for attachment to a collimator, an aiming ring or a connection body.

26. The receptor holding device of claim 21 further comprising:
a pair of fins disposed proximal to said receptor holder portion, said fins being spaced from each other to create a gap between.

27. The receptor holding device of claim 26, wherein said pair of fins are substantially parallel.

28. (canceled)

29. (canceled)

30. The receptor holding device of claim 26, wherein said gap is sized to space the two arches of teeth of a human apart.

31. The receptor holding device of claim 21 wherein said elongated arm and said receptor holder comprise separate parts prior to assembling.

32. The receptor holding device of claim 21 further comprising a bite portion disposed on said elongated arm close to said receptor holder portion.

33. The receptor holding device of claim 21 wherein said attachment portion comprises features for mating with complementary features of said interface.
